(19) United States
(12) Patent Application Publication
(10) Pub. No.: US 2010/0025619 A1
(43) Pub. Date: Feb. 4, 2010

(54) Method for heating and cooling using fluoroether compounds, compositions suitable therefore and their use

(75) Inventors: Marcello Riva, Hannover (DE); Christoph Meurer, Hannover (DE); Felix Flohr, Hildesheim (DE); Helge Rau, Burgdorf (DE)

Correspondence Address: Solvay;c/o B. Ortego - IAM-NAFTA, 3333 Richmond Avenue, Houston, TX, 77098-3099, US

(73) Assignee: SOLVAY FLUOR GMBH, Hannover (DE)

(21) Appl. No.: 12/373,032

(22) PCT Filed: Jul. 11, 2007
(86) PCT No.: PCT/EP2007/057133

§ 371 Date: Jan. 9, 2009

Related U.S. Application Data
(60) Provisional application No. 60/884,734, filed on Jan. 12, 2007.

(30) Foreign Application Priority Data
Jul. 12, 2006 (EP) 06117075.9
Sep. 25, 2006 (EP) 06121210.6
Nov. 29, 2006 (EP) 06125044.5
Jul. 5, 2007 (EP) 07111869.9

(51) Int. Cl. C09K 5/00; C07C 43/15

(52) U.S. Cl. 252/67; 568/685

(57) ABSTRACT

Compounds of general formula (I) CₓF_{y}—O—CF═CFW wherein x is 1, 2, 3, 4, 5 or 6, y=2x+1, and wherein W is F, CF₃, C₂F₅, C₃F₇, or formula (II), CₓF_{y}—O—CX═CYZ wherein x and y have the meaning given above, X stands for H or F, Y stands for H or F and Z stands for H, F. CF₃ or C₂F₅ with the proviso that at least one of X, Y or Z are H and that not more than 2 hydrogen atoms are contained in the compound of general formula (II), are suitable as or part of refrigerants, solvents, blowing agents, fire extinguishants, ORC liquids, heat transformer liquids, heat pipe liquids or aerosol-producing gases. Preferred co-refrigerants are CF₃I, HFC-134a or one or more pentafluoropropenes.

The present invention relates to methods for heating and cooling, a composition of matter based on certain unsaturated perfluoroether compounds, and the use of such unsaturated perfluoroether compounds and the composition of matter.

The use of fluorinated compounds such as CF₃I, 1,1-difluoroethane (HFC-152a) or tetrafluoropropene as fluids for heating and cooling is known from US 2005/0233923. WO 2005/021675 discloses mixtures of HFC-152a and CO₂ as substitute for 1,1,1,2-tetrafluoroethane (HFC-134a), especially in mobile air conditioning systems.

There is a need for methods of heating and cooling using novel refrigerants with advantageous properties. There is also still need for composition of matter, especially suitable for use in refrigeration, with a low GWP and, preferably, low toxicity and, preferably, low flammability or non-flammability, respectively.

It is an object of the present invention to provide a novel method for heating and cooling. Another object of the present invention is to provide novel compositions of matter suitable especially for refrigeration, but also for other purposes, such as solvent applications, heat transfer, heat pipes, ORC (organic Rankine cycle) applications, fire extinction, foam blowing, or aerosol generation. A preferred object of the present invention is to provide a composition of matter with low flammability or no flammability at all. Another object of the present invention is to provide a composition of matter with a GWP (global warming potential) preferably lower than 150, more preferably lower than 140, especially preferably equal to or lower than 120. These and other objects are met by the present invention.

One aspect of the present invention concerns a method for heating and cooling. The method according to the invention for heating or cooling operates with a refrigerant comprising of one or more compounds of the general formula (I)

CₓF_{y}—O—CF═CFW (I)

wherein x is 1, 2, 3, 4, 5 or 6 and y=2x+1, and wherein W is F, CF₃, C₂F₅, C₃F₇
or of general formula (II)

CₓF_{y}—O—CX═CYZ (II)

wherein x and y have the meaning given above, X stands for H or F, Y stands for H or F and Z stands for H, F, CF₃ or C₂F₅ with the proviso that at least one of X, Y or Z are H and not more than 2 hydrogen atoms are contained in the compound of general formula (II). The term “comprising” includes the meaning “consisting of”.

Preferred compounds of formula (I) for refrigeration purposes are CF₃—O—CF═CF₂, C₂F₅—O—CF═CF₂, i-C₃F₇—O—CF═CF₂ and n-C₃F₇—O—CF═CF₂. Preferred compounds of formula (II) for refrigeration purposes are CF₃O—CF═CHF, CF₃—O—CF═CH₂, C₂F₅—O—CF═CHF and C₂F₅—O—CF═CH₂.

The term “refrigeration” as used in the present invention includes methods which comprise condensing the refrigerant composition of the invention and thereafter evaporating it in the vicinity of a body to be cooled. Similarly, the term “refrigeration” comprises condensing the composition in the presence of a body to be heated and thereafter evaporating the composition. The heating or cooling can be effected directly or indirectly or by immersion.

The compound or compounds of formulae (I) and (II) can be used together with other refrigerants. For the sake of clarity, compounds of formula (I) or (II) will be named as “compounds A”, while the other compounds will be named as “compounds B”. The compounds B can be liquids, or they can be gaseous at standard conditions (1 bar abs, 25° C.). They can be flammable or non-flammable.

The compound A or compounds A, optionally additionally together with compounds B, can be applied together with additives. Such additives, which are named “compounds C” in the present application, improve the performance of the process. Preferred compounds C in the present invention are lubricants, stabilizers, other additives, for example, free radical scavengers, water scavengers, leak detectants, e.g. UV fluorescent dyes, corrosion inhibitors, or metal-passivating agents, for example, combinations of aminoacid derivatives and amines, or imidazoles, benzimidazoles, pyrazoles or triazoles, or antioxidants, for example, secondary arylamines, phenyl naphtylamines, diphenylamines, or hindered phenolics, for example, 2-t-butylphenol, 2,6-di-t-butylphenol or 4-methyl.2,6-di-t-butylphenol. Lubricants, stabilizers and other additives suitable for this embodiment are i.a. those that will be described in more details later for a preferred embodiment wherein the refrigerant is non-flammable.

In the following, most preferred kind of compounds C, stabilizers and lubricants, are described in detail.

Suitable stabilizers are disclosed in WO 2005/233923. For the sake of completeness, the passages relating to the stabilizers are repeated here with slight modifications.

Any of a variety of compounds suitable for stabilizing the compositions of the present invention may be used, for example phenol compounds and epoxide compounds. Examples of certain preferred stabilizers include stabilizer compositions comprising at least one phenol composition and/or at least one epoxide selected from the group consisting of aromatic epoxides, alkyl epoxides, alkenyl epoxides, and combinations of two or more thereof.

Any of a variety of phenol compounds is suitable for use in the present compositions. While applicants do not wish to be bound by or to any theory of operation, it is believed that the present phenols act as radical scavengers in the compositions and thereby tend to increase the stability of such compositions. As used herein the term “phenol compound” refers generally to any substituted or unsubstituted phenol. Examples of suitable phenol compounds include phenols comprising one or more substituted or unsubstituted cyclic, straight-chain, or branched aliphatic substituent group, such as, alkylated monophenols including for example: 2,6-di-tert-butyl-4-methylphenol; 2,6-di-tert-butyl-4-ethylphenol; 2,4-dimethyl-6-tert-butylphenol; tocopherol; hydroquinone and alkylated hydroquinones including: t-butyl hydroquinone; other derivatives of hydroquinone; hydroxylated thiodiphenyl ethers including for example: 4,4′-thiobis(2-methyl-6-tert-butylphenol); 4,4′-thiobis(3-methyl-6-tert-butylphenol); 2,2′-thiobis(4-methyl-6-tert-butylphenol); alkylidene-bisphenols including for example: 4,4′-methylenebis(2,6-di-tert-butylphenol); 4,4′-bis(2,6-di-tert-butylphenol; derivatives of 2,2- or 4,4-biphenyldiols; 2,2′-methylenebis(4-ethyl-6-tertbutylphenol); 2,2′-methylenebis(4-methyl-6-tert-butylphenol); 4,4,-butylidenebis(3-methyl-6-tert-butylphenol); 4,4,-isopropylidenebis(2,6-di-tert-butylphenol); 2,2′-methylenebis(4-methyl-6-nonylphenol); 2,2′-isobutylidenebis(4,6-dimethylphenol); 2,2′-methylenebis(4-methyl-6-cyclohexylphenol), 2,2- or 4,4-biphenyldiols including 2,2′-methylenebis(4-ethyl-6-tertbutylphenol), butylated hydro hydroxy toluene (BHT), bisphenols comprising heteroatoms including for example: 2,6-di-tert-.alpha.-dimethylamino-p-cresol; 4,4-thiobis(6-tert-butyl-m-cresol); acylaminophenols; 2,6-di-tert-butyl-4(N,N′-dimethylaminomethylphenol); sulfides including for example: bis(3-methyl-4-hydroxy-5-tert-butylbenzyl) sulfide; bis(3,5-di-tert-butyl-4-hydroxybenzyl)sulfide; as well as, phenolic UV absorb and light stabilizers. Certain preferred phenols include alkylated monophenols such as tocopherol, BHT, hydroquinones. Certain particularly preferred phenols include tocopherol. Most phenols are commercially available. A single phenol compound and/or mixtures of two or more phenols may be used in the present compositions.

Any of a variety of epoxides is suitable for use in the compositions of the present invention. It is believed that the epoxides of the present invention act as acid scavengers in the compositions and thereby tend to increase the stability of such compositions. A single aromatic epoxide and/or mixtures of two or more aromatic epoxides may be used in the present compositions.

Examples of suitable aromatic epoxides include those defined by the formula (III) below:

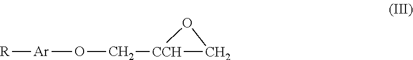

wherein: R is hydrogen, hydroxyl, alkyl, fluoroalkyl, aryl, fluoroaryl, or

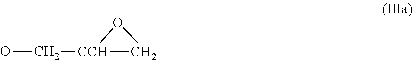

and Ar is a substituted or unsubstituted phenylene or napthylene moiety. Certain preferred aromatic epoxides of Formula (III) include those wherein Ar is phenylene or phenylene substituted with one or more substituents including for example alkyls, alkenyls, alkynyls, aryls, alkylaryls, halogens, halogenated alkyls, halogenated alkenyls, halogenated alkynyls, halogenated aryls, halogenated arylalkyls, hydroxyls, heteroatom moieties. Examples of suitable compounds of Formula I(III) wherein Ar is an unsubstituted or substituted phenylene include, for example, butylphenylglycidyl ether; pentylphenylglycidyl ether; hexylphenylglycidyl ether; heptylphenylglycidyl ether; octylphenylglycidyl ether; nonylphenylglycidyl ether; decylphenylglycidyl ether; glycidyl methyl phenyl ether; 1,4-diglycidyl phenyl diether; 4-methoxyphenyl glycidyl ether; derivatives thereof. Certain other preferred aromatic epoxides of Formula (III) include those wherein Ar is napthylene or napthylene substituted with one or more substituents including for example alkyls, alkenyls, alkynyls, aryls, alkylaryls, halogens, halogenated alkyls, halogenated alkenyls, halogenated alkynyls, halogenated aryls, halogenated arylalkyls, hydroxyls and heteroatom moieties. Examples of suitable compounds of formula (III) wherein Ar is an unsubstituted or substituted napthylene include, for example, naphthyl glycidyl ether; 1,4-diglycidyl naphthyl diether; derivatives thereof. Examples of other suitable aromatic epoxides include bisoxiranes, for example, 2,2′[[[5-heptadecafluorooctyl]1,3-phenylene]-bis[[2,2,2-trifluoromethyl]ethylidene]-oxymethylene]bisoxirane. In certain preferred embodiments, the aromatic epoxides for use in the present invention comprise an epoxide of Formula (III) wherein Ar is phenylene, substituted phenylene, napthylene, or substituted napthylene. More preferably, the aromatic epoxides comprise an epoxide of Formula (III) wherein Ar is phenylene or substituted phenylene. Examples of certain more preferred aromatic epoxides include for example butylphenyl glycidyl ether. Any of a variety of alkyl and/or alkenyl epoxides is suitable for use in the present compositions. Examples of suitable alkyl and alkenyl epoxides include those of Formula (IV):

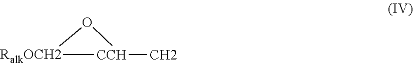

wherein Rₐₗₖ is a substituted or unsubstituted alkyl or alkenyl group. Certain preferred epoxides of Formula (IV) comprise alkyl epoxide compounds wherein Rₐₗₖ is an alkyl group having from about 1 to about 10 carbon atoms, more preferably from about 1 to about 6 carbon atoms, and wherein the alkyl may be unsubstituted or further substituted with one or more substituents including alkyls, alkenyls, alkynyls, aryls, alkylaryls, halogens, halogenated alkyls, halogenated alkenyls, halogenated alkynyls, halogenated aryls, halogenated arylalkyls, hydroxyls and heteroatom moieties. Examples of such preferred alkyl epoxides of Formula (IV) include n-butyl glycidyl ether, isobutyl glycidyl ether and hexanediol diglycidyl ether as well as, for example, fluorinated and perfluorinated alkyl epoxides. Certain more preferred alkyl epoxides comprise for example hexanediol diglycidyl ether. Certain other preferred epoxides of Formula (IV) comprise alkenyl epoxide compounds wherein Rₐₗₖ is an alkenyl group having from about 1 to about 10 carbon atoms, more preferably from about 1 to about 6 carbon atoms, and wherein the alkenyl may be unsubstituted or further substituted with one or more substituents including alkyls, alkenyls, alkynyls, aryls, alkylaryls, halogens, halogenated alkyls, halogenated alkenyls, halogenated alkynyls, halogenated aryls, halogenated arylalkyls, hydroxyls and heteroatom moieties. Examples of such preferred alkenyl epoxides of Formula (IV) include, for example, allyl glycidyl ether, fluorinated and perfluorinated alkenyl epoxides. More preferred alkenyl epoxides include, for example, allyl glycidyl ether. A single alkyl epoxide or alkenyl epoxide and/or combinations of two or more thereof may be used in the present compositions. In certain other preferred embodiments, the alkyl epoxide for use as an acid scavenger in the present composition comprises polypropylene glycol diglycidyl ether. Examples of polypropylene glycol diglycidyl ethers suitable for use in the present invention includes the ether available commercially from SACHEM, Europe. In addition, in certain embodiments, the epoxide for use in the present invention comprises combinations of two or more aromatic, alkyl, and/or alkenyl substituents. Such epoxides are referred to generally as “multisubstituted epoxides”. According to certain preferred embodiments, the stabilizer for use in the present invention comprises a combination of at least one phenol compound and at least one aromatic, alkyl, or alkenyl epoxide. Examples of suitable combinations include stabilizers comprising, for example, tocopherol and allyl glycidyl ether, BHT and glycidyl butyl ether. Certain particularly preferred combinations include stabilizers comprising, for example, tocopherol and allyl glycidyl ether. Any suitable relative amount of the at least one phenol compound and the at least one aromatic, alkyl, or alkenyl epoxide may be used in the preferred stabilizers. For example, the weight ratio of phenol compound(s) to aromatic or fluorinated alkyl epoxide(s) can be varied from about 1:99 to about 99:1. In certain preferred embodiments, the weight ratios of phenol compound(s) to aromatic, alkyl, alkenyl, multisubstituted, or fluorinated alkyl epoxide(s) is from about 30 to about 1, more preferably from about 7 to about 1, more preferably from about 2 to about 1, and even more preferably about 1:1.

Any suitable effective amount of stabilizer may be used in the compositions of the present invention. The stabilizers mentioned above are especially suitable for compositions comprising CF₃I (trifluoroiodomethane). As used herein, the term “effective stabilizing amount” especially refers to an amount of stabilizer of the present invention which, when added to a composition, especially if it contains trifluoroiodomethane, results in a stabilized composition wherein the composition, especially the trifluoroiodomethane therein, degrades more slowly and/or to a lesser degree relative to the original composition, under the same, or similar, conditions. In certain preferred embodiments, an “effective stabilizing amount” of stabilizer comprises an amount which, when added to a composition, especially if it contains trifluoroiodomethane, results in a stabilized composition which degrades more slowly and/or to a lesser degree relative to the original composition under the conditions of at least one, or both, of the standards tests SAE J1662 (issued June 1993) and/or ASHRAE 97-1983R; especially, this holds true for trifluoroiodomethane if it is comprised therein. In certain more preferred embodiments, an “effective stabilizing amount” of stabilizer comprises an amount which, when added to a composition comprising trifluoroiodomethane, results in a composition having a stability that is at least as good as, if not better, than the stability of a comparable composition comprising dichlorodifluoromethane (R-12) in mineral oil, under at least one of the standard tests SAE J1662 (issued June 1993) and/or ASHRAE 97-1983R. Certain preferred effective amounts of stabilizer for use in the present invention comprise from about 0.001 to about 10, more preferably from about 0.01 to about 5, even more preferably from about 0.3 to about 4 weight percent, and even more preferably from about 0.3 to about 1 weight percent based on the total weight of the composition, or, if trifluoroiodomethane is contained, based on the total weight of trifluoroiodomethane in the composition of the present invention.

Another especially suitable type of stabilizers are unsaturated hydrocarbons, especially terpenes, for example, monoterpenes, diterpenes and sesquiterpenes. Preferred terpenes which can be applied as a stabilizer are citral, citronellal, citronellol, limonene, dipentene, menthol, terpinene, terpinolene, sylvestrene, sabinene, menthadiene, zingiberene, ocimene, myrcene, α-pinene, β-pinene, turpentine, camphor, phytol, squalene, and lycopene. Of course, if desired, mixtures of 2 or more terpenes can be used as stabilizer. Preferably, the terpene is comprised in an amount of equal to or more than 0.1%, especially preferably equal to or more than 0.2% by weight of the total weight of the composition. Preferably, the terpene is comprised in an amount equal to or less than 3%, preferably 2% by weight of the total weight of the composition. In

In certain preferred embodiments, the compositions of the present invention further comprise a lubricant as compound C. In WO 2005/233923, suitable lubricants are mentioned. For the sake of completeness, the respective passages are repeated here.

Any of a variety of conventional lubricants may be used in the compositions of the present invention, optionally in the presence of solubility compatibilizers. An important requirement for the lubricant is that, when in use in a refrigerant system, there must be sufficient lubricant returning to the compressor of the system such that the compressor is lubricated. Thus, suitability of a lubricant for any given system is determined partly by the refrigerant/lubricant characteristics and partly by the characteristics of the system in which it is intended to be used. Examples of suitable lubricants include mineral oil, for example, paraffins, naphthenes, or aromatics, or synthetic oils, for example, arylalkyls, e.g. alkyl benzenes, polyol esters, polyalkylene glycols, PAG oils, phosphate esters, dibasic acid esters, fluoroesters and polyvinylethers. Mineral oil, which comprises paraffin oil or naphthenic oil, is commercially available. Commercially available mineral oils include Witco LP 250 (registered trademark) from Witco, Zerol 300 (registered trademark) from Shrieve Chemical, Sunisco 3GS from Witco, and Calumet R015 from Calumet. Commercially available alkyl benzene lubricants include Zerol 150 (registered trademark). Alkyl benzene lubricants with a kinematic viscosity at 40° C. in the range of 46 mm²/s, for example, Fuchs Reniso S46F, are also very suitable. Commercially available esters include neopentyl glycol dipelargonate which is available as Emery 2917 (registered trademark) and Hatcol 2370 (registered trademark). Other useful esters include phosphate esters, dibasic acid esters, and fluoroesters. Preferred lubricants include polyalkylene glycols and esters. Certain more preferred lubricants include polyalkylene glycols. A very suitable PAG oil is ND8 PAG of Denso.

The amount of lubricant is selected so that the apparatus works reliably. The amount of lubricant may be comprised in the refrigerant composition in the range of 1 to 35% by weight of the total refrigerant composition (including compounds A, compounds B, and compounds C, for example, stabilizers, lubricants, other additives). A preferred range is 5 to 30% by weight.

The composition may further comprise other additives as compound C. Metal passivators and corrosion inhibitors such as those described above can be comprised in the range of 0.01 to 5%, preferably 0.05 to 2% by weight of the total composition. Other additives, for example, antioxidants can each be present in the range of 0.01 to 5%, preferably 0.05 to 2% by weight of the total composition. Of course, if desired, the refrigerant may comprise two or more different kinds of compounds C, e.g. stabilizers together with lubricants and/or corrosion inhibitors. Also, solubility compatibilizers, for example, polyoxyalkylene glycol ethers, amides, ketones, nitriles, chlorocarbons, arylethers, 1,1,1-trifluoroalkanes, fluoroethers or esters may be present to optimize solubility of the refrigerant in the selected oil.

One embodiment of the invention concerns a method for refrigeration in situations where flammability is of no concern. This may be the case in stationary refrigeration apparatus such as freezers where leakage of the refrigerant caused e.g. by mechanical impact is not to be expected, in situations where any leakage does not lead to danger of fire. The term “flammability” in the context of the present invention is defined by ASTM standard E-681. This standard describes how to evaluate the lower and upper flammability ranges, performed in an open glass bowl with electric ignition.

In this variant of the embodiment where flammability is of no concern, the compounds of formula (I) or (II) can be used as such. If desired, they can be used in the form of mixtures of two or more of compounds of formula (I) and/or (II). They also can be used together with compounds B which are flammable themselves, or which are non-flammable, but applied in an amount less than the amount needed to render the refrigerant mixture non-flammable.

Such additional compound B or compounds B can for example be selected from flammable compounds. For example, linear, branched or cyclic hydrocarbons (HC), e.g. with 1 to 8 carbon atoms, for example propane, cyclopropane, n-butane, i-butane, or the pentanes, for example, 2-methylbutane, n-pentane or cyclopentane, are suitable as compounds B. Ethers, especially dialkylethers, e.g. dimethylether, fluorinated ethers with lower fluorine content or fluorinated thioethers, for example, CF₃—S—CF₃ are also suitable as compound B. Another class of suitable compounds B are C1 to C6 saturated or C2 to C6 unsaturated hydrofluorocarbons (HFC) for example fluoromethane, difluoromethane, fluoroethane, 1,1-difluoroethane or 1,1,1-trifluoroethane, the fluoropropenes, e.g. 2-fluoropropene, trifluoropropenes, tetrafluoropropenes, preferably trans-1,1,1,3-tetrafluoropropene, ketones, for example, acetone. The present invention is intended to include all single configurational isomers, single stereomers and mixtures thereof, e.g. of the trifluoropropenes, tetrafluoropropenes and pentafluoropropenes. For example, 1,3,3,3-tetrafluoropropene (HFC-1234ze) is meant to represent the cis-isomer, the trans-isomer and any mixtures thereof in any ratio. 1,2,3,3,3-pentafluoropropene (HFC-1225ye) represents the cis-isomer (Z isomer), the trans-isomer (E isomer) and any mixtures thereof in any ratio.

Also in this embodiment where flammability is of no concern, non-flammable compounds can be applied, e.g. in amounts not sufficient to render the composition non-flammable. CF₃I, perfluorocarbons, preferably with 2 to 6 carbon atoms, e.g. hexafluorocyclopropane, and higher-fluorinated saturated or unsaturated hydrofluorocarbons, fluorinated ketones, for example, perfluoro-(methyl-isopropyl ketone), perfluoro-(ethyl-isopropyl ketone), saturated fluoroethers, for example, trifluoromethyl-difluoromethylether (E-125), trifluoromethyl-fluoromethylether (E-134a) or trifluoromethyl-methylether (E-143a), or carbon dioxide can, for example, be applied. Very suitable saturated non-flammable compounds are selected from C1 to C4 hydrofluorocarbons wherein the number of H atoms is lower than the number of F atoms, especially from trifluoromethane, HFC-134, HFC-134a, HFC-125, the pentafluoropropanes, for example, 1,1,2,2-pentafluoropropane (HFC-245cb) or 1,1,1,3,3-pentafluoropropane (HFC-245fa), hexafluoropropanes, for example, 1,1,1,3,3,3-hexafluoropropane (HFC-236fa) or heptafluoropropanes, for example, 1,1,1,2,3,3,3-heptafluoropropane (HFC-227ea), CO₂ and especially CF₃I. Other preferred non-flammable compounds which can be used as compounds B are higher-fluorinated non-flammable unsaturated C2 to C4 hydrofluorocarbons, especially pentafluoropropenes. The embodiment wherein compounds of formula (I) or (II) are applied together with unsaturated hydrofluorocarbons will be explained in detail later.

It has been found that mixtures comprising CF₃—O—CF═CF₂ and HFC-134a form azeotropes. For example, at 0° C., mixtures consisting of about 20% by weight to about 60% by weight of CF₃—O—CF═CF₂ and the balance to 100% by weight being HFC-134a form an azeotrope with a minimum boiling point. The pressure of the azeotrope at 0° C. is about 3.57 bars (absolute). Such azeotropes have the advantage that the composition in the vapor phase and the composition in the condensed phase are identical. Consequently, the composition in the condensed phase does not change even if deliberately part of the vapor phase is removed from the refrigeration apparatus or leaves the refrigeration apparatus inadvertently, e.g. through a leakage. These azeotropes can of course be mixed with other refrigerant components, for example, carbon dioxide, hydrocarbons or CF₃I, and/or with one or more of the additives mentioned above to provide a refrigerant.

It also has been found that mixtures of the E isomer, the Z isomer or the mixtures of E and Z isomer of HFC-1225ye in any ratio, and perfluoro-methylvinyl ether form an azeotrope-like mixture. Azeotrope-like mixtures which have an ODP of 0 and a GWP lower than 10, are a preferred embodiment of the present invention and are described in the following.

An azeotrope-like mixture in the frame of the present invention is a mixture of two or more fluids having a vapor composition substantially equal to that of the liquid and which undergoes phase changes without substantially modifying its composition and temperature. According to the present invention, a mixture is azeotrope-like when, after evaporation at a constant temperature of 50% of the initial liquid mass, the percent variation of the vapor pressure between that of the initial mixture and that of the final mixture results lower than about 10%. See on the matter the paper of D. A. Didion and D. B. Bivens in Int. J. of Refrigeration 13 (1990), pages 163 to 175.

It was found that binary mixtures of perfluoro-methylvinyl ether and the E isomer, the Z isomer or any mixtures of E and Z isomers of HFC-1225ye form an azeotrope-like mixture in a broad range.

Tests have shown that PVME permeates through plastic material much slower than either pentafluoropropene. On the other hand, the thermodynamic behaviour of mixtures with a comparably low content of PVME, e.g. in the range of 10% by weight, have very good thermodynamic properties. Additionally, for certain compositions, an azeotropi-like behaviour exists which is advantageous for some applications. So, different embodiments of preferred compositions of PVME and the pure or mixed isomers of HFC-1225ye exist. Mixtures of perfluoromethylvinyl ether and the E isomer, the Z isomer or any mixtures of the E and Z isomer of HFC-1225ye with a content of equal to or more than 8% by weight, more preferably equal to or more than 10% by weight, very preferably equal to or more than 15% by weight of perfluoro-methylvinyl ether are very advantageous. Advantageously, the content of perfluoro-methylvinyl ether is equal to or lower than 30% by weight. These mixtures have an ODP of zero and a GWP equal to or lower than 10.

In one embodiment of the invention, a very preferred azeotrope-like mixture consists 15 to 25% by weight of perfluoro-methylvinyl ether and 75 to 85% by weight of the E isomer, the Z isomer or any mixtures of the E and Z isomers of HFC-1225ye.

A mixture even comprising 20% by weight of perfluoro-methylvinyl ether, the remainder to 100% by weight being the E isomer, the Z isomer or any mixture of the E and the Z isomer of HFC-1225ye, is non-flammable. It has a temperature glide of about 1 to 2 K at p_{S} 2 bar. The term “P_{S}” denotes the saturation pressure. The temperature glide can be utilized to enhance performance.

Another embodiment of the present invention are ternary compositions comprising the azeotrope-like mixture of perfluoro-methylvinyl ether and the E isomer, Z isomer of HFC-1225ye or the mixtures thereof, and, as the third component, a compound suitable as refrigerant selected from the group consisting of perfluoro-ethylvinylether, perfluoro-propylvinylether, perfluoro-methyl-methylvinylether, HFC-1234ze, HFC-1234yf, HFC-1234ye, HFC-1243zf, HFC-32, HFC-125, HFC-134, HFC-134a, HFC-143a, HFC-152a, HFC-161, HFC-227ea, HFC-236ea, HFC-236fa, HFC-245fa, HFC-365mfc, propane, n-butane, isobutane, 2-methylbutane, n-pentane, cyclopentane, dimethylether, CF₃SCF₃, CO₂ and CF₃I. Preferred azeotrope-like mixtures of FHC-1225ye and perfluoro-methylvinyl ether which are comprised in these ternary compositions are described above.

If the ternary compositions of perfluoro-methylvinyl ether, HFC-1225ye and the third component are intended to be a substitute for HFC-134a, the third component preferably has a boiling point which is lower than that of the azeotrope-like mixture. For example, compounds with a boiling point in the range of −25 to −100° C. are advantageously used as a third component.

In ternary compositions, the content of the azeotrope-like mixture as described above can be higher or equal to 1% by weight, preferably equal to or higher than 10% by weight, still more preferably equal to or higher than 20% by weight, especially equal to or higher than 30% by weight. Very preferably, the content of the azeotrope-like mixture is equal to or higher than 50% by weight. The content of the azeotrope-like mixture in ternary compositions can be equal to or lower than 99% by weight, preferably equal to or lower than 97% by weight. The azeotropic mixture and of the third component add up to 100% by weight. It is assumed that the composition has a low toxicity.

If the ternary mixture is intended to be a substitute for HFC-134a, the amount of the azeotrope-like mixture of perfluoro-methylvinyl ether and HFC-1225ye are selected such that the vapor pressure curve is similar to that of HFC-134a.

Very often, compounds B with a boiling point in the range of ±20° C., preferably in the range of ±10° C., of the compound or compounds A are advantageous.

In this embodiment, the content in % by weight of the compound A, or the sum of compounds A if more than one of compounds a is comprised, and the compound B, or the sum of compounds B in more than one compound B is comprised, in the refrigerant (which may comprise compounds A and compounds B or compounds A, compounds B and compounds C) is preferably as follows. The amount of compound A (or their sum) preferably is equal to or higher than 1% by weight. The preferred amount is equal to or higher than 5% by weight, still more preferably equal to or higher than 10% by weight, especially preferably equal to or higher than 20% by weight and most preferably, the amount of compound A is equal to or higher than 30% by weight. The amount of compound A (or of their sum if more than one compound A is comprised) is preferably equal to or lower than 99% by weight, preferably equal to or lower than 95% by weight. Still more preferably, the amount of compound(s) A is equal to or lower than 90% by weight, especially equal to or lower than 80% by weight.

The amount of compound B preferably is equal to or higher than 1% by weight. The preferred amount is equal to or higher than 5% by weight, still more preferably equal to or higher than 10% by weight, especially preferably equal to or higher than 20% by weight and most preferably, the amount of compound B is equal to or higher than 30% by weight. The amount of compound(s) B is preferably equal to or lower than 99% by weight, preferably equal to or lower than 95% by weight. Still more preferably, the amount of compound(s) B is equal to or lower than 90% by weight, especially equal to or lower than 80% by weight.

As mentioned above, CF₃I is one of the preferred compounds.

In another preferred embodiment, preferred compounds B are saturated HFCs, especially HFC-227ea or HFC-134a. In particular preferred is a binary mixture consisting essentially of CF₃—O—CF═CF₂ and HFC-227ea, or a binary mixture consisting essentially of CF₃—O—CF═CF₂ and HFC-134a.

Preferably, the content of HFC-227ea in such binary mixtures is equal to or less than 5% by weight. Preferably, the content of HFC-227ea is equal to or higher than 1% by weight. Preferably, the content of CF₃—O—CF═CF₂ is equal to or higher than 95% by weight of the binary composition with HFC-227ea. Especially preferably, the content of CF₃—O—CF═CF₂ is equal to or lower than 99% by weight of the binary composition with HFC-227ea.

For mixtures with HFC-134a, preferably, the content of HFC-134a in such binary mixtures is equal to or less than 10% by weight. Preferably, the content of HFC-134a is equal to or higher than 1% by weight. Preferably, the content of CF₃—O—CF═CF₂ is equal to or higher than 90% by weight of the binary composition with HFC-134a. Preferably, the content of CF₃—O—CF═CF₂ is equal to or lower than 99% by weight of the binary composition with HFC-134a.

Examples of suitable binary compositions of CF₃—O—CF═CF₂ and given in the following table:

| Content of CF₃—O—CF═CF₂ | Content of HFC-134a or |
|---|---|
| [% by weight] | HFC-227ea [% by weight] |
| 99 | HFC-134a; 1 |
| 97 | HFC-134a; 3 |
| 95 | HFC-134a; 5 |
| 93 | HFC-134a; 7 |
| 92 | HFC-134a; 8 |
| 91.5 | HFC-134a; 8.5 |
| 91 | HFC-134a; 9 |
| 90 | HFC-134a; 10 |
| 99 | HFC-227ea; 1 |
| 98 | HFC-227ea; 2 |
| 97 | HFC-227ea; 3 |
| 96 | HFC-227ea; 4 |
| 95 | HFC-227ea; 5 |

Compositions of compounds of formula (I) with a content equal to or less than 11.5% by weight of HFC-134a have a GWP of less than 150. Compositions with a content equal to or less than 10.7% by weight of HFC-134a have a GWP of less than 140. Such compositions with a content equal to or less than 9.5% by weight of HFC-134a have a GWP of less than 120 and are preferred. Compositions of compounds of formula (I) with HFC-134a in which HFC-134a is comprised in a range of 7 to 11.5% by weight, preferably 7 to 10.7% by weight, especially preferably 7 to 9.5% by weight have the advantage that their GWP is lower than 150, lower than 140 and even lower than 120 while having advantageous properties. A highly suitable composition is a mixture of CF₃—O—CF═CF₂ and HFC-134a with a content of HFC-134a in the range of 8.0% to 9% by weight.

If CO₂ is comprised additionally, the amount is preferably 0.1% by weight, more preferably 1% by weight or more of the refrigerant. If comprised, the amount of CO₂ is preferably equal to or lower than 15% by weight.

The advantage of the compositions mentioned above is i.a. that the compounds of formula (I) and (II) are expected to have a very low GWP and, for example, that compounds B have no ozone depletion potential. The preferred compound, CF₃—O—CF═CF₂, has a low acute toxicity like, many of compounds B.

In a preferred embodiment, the compound A or compounds A of formula (I) are applied together with at least one non-flammable compound B which is comprised at least in an amount which eliminates the flammability of the thus formed composition. Preferred non-flammable compounds B are gaseous or liquid at standard conditions. They are preferably selected from non-flammable compounds of the group consisting of CF₃I, perfluorocarbons and saturated hydrofluorocarbons, especially with 1 to 5 carbon atoms, unsaturated hydrofluorocarbons, especially with 2 to 5 carbon atoms, fluorinated ketones, especially those with 3 to 9 carbon atoms, for example, perfluoro-(methyl-isopropyl ketone), perfluoro-(ethyl-isopropyl ketone), or polyfluoronated ketones with at most 2 hydrogen atoms, saturated fluoroethers, for example, trifluoromethyl-difluoromethylether (E-125), trifluoromethyl-fluoromethylether (E-134a) or trifluoromethyl-methylether (E-143a), and carbon dioxide.

According to one preferred embodiment of performing refrigeration with non-flammable compositions, very suitable non-flammable compounds B are selected from CF₃I and among saturated hydrofluorocarbons, preferably with 1 to 4 carbon atoms, especially among the group consisting of trifluoromethane, HFC-134, HFC-134a, HFC-125, the pentafluoropropanes, for example, 1,1,1,3,3-pentafluoropropane (HFC-245fa), hexafluoropropanes, for example, 1,1,1,3,3,3-hexafluoropropane (HFC-236fa) or heptafluoropropanes, for example, 1,1,1,2,3,3,3-heptafluoropropane (HFC-227ea), and CO₂.

Especially preferred is CF₃I as saturated compound B.

A very preferred method of refrigeration in accordance with this embodiment is performed with a composition comprising CF₃—O—CF═CF₂ and CF₃I. Such compositions may comprise CO₂. If CO₂ is comprised, it is preferably comprised in an amount of up to 15% by weight, relative to the total weight of the composition. Of course, also these preferred compositions may comprise one or more of compounds C in amounts as outlined above.

The lower limit of the content of the non-flammable compound B is selected such that the refrigerant (including compounds C, if comprised) is non-flammable. That minimum content can be easily determined by trials using standard equipment according to ASTM E681.

In this embodiment, the amount of compound A, preferably CF₃—O—CF═CF₂ in the refrigerant (which may comprise compounds A, compounds B and optionally compounds C) preferably is equal to or higher than 20% by weight. A more preferred amount is equal to or higher than 30% by weight; still more preferred equal to or higher than 40% by weight, especially equal to or higher than 50% by weight. Very preferably, the amount is equal to or higher than 60% by weight. Preferably, the amount of compound A, preferably CF₃—O—CF═CF₂, is equal to or lower than 80% by weight, more preferred equal to or lower than 70% by weight.

The preferred amount of compound B, preferably CF₃I, is equal to or higher than 20% by weight, relative to the total weight of compounds A and B of the refrigerant, more preferably, it is equal to or higher than 30% by weight. If desired, the content of CF₃I can be still higher, for example, equal to or higher than 40% by weight. Preferably, the amount of compound B, which preferably is CF₃I, is equal to or lower than 80% by weight; more preferred equal to or lower than 70% by weight, still more preferably equal to or lower than 60% by weight, especially preferably equal to or lower than 50% by weight, still more preferably equal to or lower than 40% by weight.

In a particular embodiment, for compositions of CF₃—O—CF═CF₂ and CF₃I, the content of CF₃—O—CF═CF₂ is preferably equal to or greater than 50% by weight and the content of CF₃I is preferably lower than 50% by weight but sufficient to impart non-flammability, according to the ASTM E681 standard (preferably the version of 2001).

In a preferred embodiment, constituents and their amounts are chosen such that the refrigerant has a GWP lower than 150. The GWP can be determined according to the method devised by Scientific Assessment of Stratospheric Ozone: 1989” sponsored by the U.N. Environment Programme. The general definition is

GWP=Calculated IR forcing due to agent/Emission rate (steady state) of agent divided by the same parameters for CFCl₃.

In a preferred embodiment, the GWP of the composition is lower than 140. In an especially preferred embodiment, the GWP of the compositions is equal to or lower than 120. In a most preferred embodiment, the GWP is 40 or less, most preferably, equal to or less than 10.

The invention will now be further explained in view of one of the preferred embodiments wherein CF₃—O—CF═CF₂ and CF₃I are comprised in the refrigerant.

In one embodiment, the components A and B of the refrigerant consist essentially of CF₃—O—CF═CF₂ as compound A and CF₃I as compound B, respectively. Suitable gaseous mixtures, optionally liquefied under pressure, calculated for the total weight of both compounds, are given in the following table 1:

| Content of CF₃—O—CF═CF₂ | Content of CF₃I |
|---|---|
| [% by weight] | [% by weight] |
| 10 | 90 |
| 15 | 85 |
| 18 | 82 |
| 20 | 80 |
| 30 | 70 |
| 40 | 60 |
| 45 | 55 |
| 50 | 50 |
| 55 | 45 |
| 60 | 40 |
| 65 | 35 |

It is assumed that these mixtures are quasi-azeotropes. Compositions comprising of CF₃I in the lower range (e.g. with 35, 40, 45 or 50% by weight of C₃FI) are preferred.

In another embodiment, the preferred non-flammable refrigerant mixtures of CF₃—O—CF═CF₂ and CF₃I may comprise further compounds B. These additional compounds B may be selected from the compounds B mentioned above, namely flammable compounds, for example, linear, branched or cyclic hydrocarbons (HC), such as propane, cyclopropane, n-butane, i-butane, the pentanes, hydrofluorocarbons (HFC) with lower fluorine substitution such as fluoromethane, difluoromethane, fluoroethane, 1,1-difluoroethane or 1,1,1-tri-fluoroethane, trifluoropropenes, tetrafluoropropenes, preferably trans-1,1,1,3-tetrafluoropropene, dialkylethers, for example, dimethylether, ketones, for example, acetone, or they may be selected from non-flammable compounds mentioned above such as perfluorocarbons and saturated or unsaturated hydrofluorocarbons, fluorinated ketones, for example, perfluoro-(methyl-isopropyl ketone), perfluoro-(ethyl-isopropyl ketone), saturated fluoroethers, for example, trifluoromethyl-difluoromethylether (E-125), trifluoromethyl-fluoromethylether (E-134a) or trifluoromethyl-methylether (E-143a), carbon dioxide. Non-flammable compounds especially suitable as additional compound B are selected from trifluoromethane, HFC-134, HFC-134a, HFC-125, the pentafluoropropanes, for example, 1,1,1,3,3-pentafluoropropane (HFC-245fa), hexafluoropropanes, for example, 1,1,1,3,3,3-hexafluoropropane (HFC-236fa) or heptafluoropropanes, for example, 1,1,1,2,3,3,3-heptafluoropropane (HFC-227ea), the pentafluoropropenes or CO₂.

The amount of the additional compound B depends on the properties. If a flammable compound B is to be included as further compound B, its amount is limited when the resulting composition is intended be non-flammable. In this case, often 50% by weight or less of CF₃—O—CF═CF₂ is substituted by the additional flammable compound B; the remaining content of CF₃I in this case provides for the non-flammability of the resulting composition. If the further compound B is a non-flammable compound, it can substitute some of the CF₃—O—CF═CF₂ and/or some of the CF₃I. Often, 50% by weight or less of CF₃—O—CF═CF₂ and/or 50% by weight or less of CF₃I is substituted.

Some suitable ternary and multiple gaseous compositions, optionally liquefied, are given in table 2 (figures denoting the content in % by weight, calculated for the total weight of the components given in the table, and giving a range for other examples of mixtures according to the present invention; the amounts always add up to 100%):

| | | Other | |
|---|---|---|---|
| CF₃—O—CF═CF₂ | CF₃I | component | Other component |
| 10 ± 5 | 85 ± 5 | CO₂: 5 ± 4 | |
| 35 ± 10 | 60 ± 10 | CO₂: 5 ± 4 | |
| 55 ± 10 | 41 ± 10 | CO₂: 4 ± 3 | |
| 54 ± 10 | 36 ± 10 | CO₂: 4 ± 3 | 1,1,1,3-tetrafluoropropene: |
| | | | 6 ± 5 |
| 60 ± 10 | 36 ± 10 | | HFC-134a: 4 ± 3 |
| 60 ± 10 | 35 ± 10 | | HFC-227ea 3 ± 2 |
| 60 ± 10 | 35 ± 10 | | HFCF-134a 3 ± 2 |
| | | | HFC-227ea 2 ± 1 |

According to another preferred embodiment performing refrigeration with non-flammable compositions, mixtures of compounds of formula (I) or (II) together with one or more non-flammable unsaturated hydrofluorocarbons as compound B are applied. In this embodiment, CF₃—O—CF═CF₂, C₂F₅—O—CF═CF₂, i-C₃F₇—O—CF═CF₂ and n-C₃F₇—O—CF═CF₂ are preferred compounds of formula (I). This embodiment will explained in detail for CF₃—O—CF═CF₂ being a very preferred compound of formula (I). The term “unsaturated fluorohydrocarbons” denotes unsaturated compounds composed of carbon, hydrogen and fluorine wherein the number of fluorine atoms is higher than the number of hydrogen atoms so that the compound is non-flammable. Preferably, these unsaturated hydrofluorocarbons have 2 to 5 carbon atoms, preferably 2 to 4, especially preferably 3 carbon atoms. By definition, they comprise at least one hydrogen atom.

Most preferred unsaturated compounds in this embodiment are the pentafluoropropenes. The pentafluoropropenes may exist as different configurational isomers, e.g. CF₃—CH═CF₂ and CF₃—CF═CHF, or stereoisomers. CF₃—CF═CHF exists, for example, in the form of (E)- and (Z) isomer. The present invention is intended to include all single configurational isomers, single stereomers and mixtures thereof. It also has to be noted that in the compositions in this application, the (Z) isomer of HFC-1225ye is the preferred isomer.

Preferred compositions comprise one or more of CF₃—O—CF═CF₂, C₂F₅—O—CF═CF₂, i-C₃F₇—O—CF═CF₂ and n-C₃F₇—O—CF═CF₂, as compounds of formula (I), and one or more of HFC-1225ye, HFC-1234ze, HFC-1234yf, HFC-1234ye and HFC-1243zf.

The compositions may also comprise the azeotrope-like compositions of the (Z)-isomer of HFC-1225ye and HFC-1234yf as described by US patent application publication 2005/0233923. Preferably, this azeotrope-like composition consists of 50 to less than 100% by weight of HFC-1234yf and from greater than 0 to less than 50% by weight of (Z)-HFC-1225ye.

Optionally, other compounds B may additionally be comprised as additional component. In this case, one or more of HFC-32, HFC-134, HFC-134a, HFC-152a, and CO₂ are preferably comprised.

The composition of matter of this embodiment may comprise the compound or compounds of formula (I) in an amount of 1 to 99% by weight and the compound B or compounds B in an amount of 99 to 1% by weight, relative to the total weight of the composition. If one or more of the unsaturated fluorohydrocarbons and one or more additional compounds B, for example, HFC-134a, HFC-152a or CO₂ are comprised, then the sum of compounds B is comprised in an amount of 99 to 1% by weight.

The following tables describe preferred binary, ternary and quaternary compositions. They also include some flammable compositions. The term “Any*” denotes any of the compounds B mentioned above, for example, linear, branched or cyclic hydrocarbons (HC), such as propane, cyclopropane, n-butane, i-butane, the pentanes, hydrofluorocarbons (HFC) with lower fluorine substitution such as fluoromethane, difluoromethane, fluoroethane, 1,1-difluoroethane or 1,1,1-tri-fluoroethane, trifluoropropenes, tetrafluoropropenes, preferably trans-1,1,1,3-tetrafluoropropene, pentafluoropropenes, dialkylethers, for example, dimethylether, ketones, for example, acetone, perfluorocarbons and saturated or unsaturated hydrofluorocarbons, fluorinated ketones, for example, perfluoro-(methyl-isopropyl ketone), perfluoro-(ethyl-isopropyl ketone), saturated fluoroethers, for example, trifluoromethyl-difluoromethylether (E-125), trifluoromethyl-fluoromethylether (E-134a) or trifluoromethyl-methylether (E-143a), carbon dioxide, especially trifluoromethane, HFC-134, HFC-134a, HFC-125, the pentafluoropropanes, for example, 1,1,1,3,3-pentafluoropropane (HFC-245fa), hexafluoropropanes, for example, 1,1,1,3,3,3-hexafluoropropane (HFC-236fa) or heptafluoropropanes, for example, 1,1,1,2,3,3,3-heptafluoropropane (HFC-227ea), the pentafluoropropenes or CO₂ The term “HFC-1225ye” denotes (Z)-HFC-1225ye, (E)-HFC-1225ye and mixtures thereof in any molar ratio.

TABLE 3
Compositions comprising CF₃—O—CF═CF₂ and HFC-1225ye

| Content of | Content of compound | |
|---|---|---|
| CF₃—O—CF═CF₂ | or compounds B [% by weight] | |
| [% by weight] | Fluoropropene | Other |
| 1-99 | HFC-1225ye; 99-1 | |
| 1-99 | (Z)-HFC-1225ye; 99-1 | HFC-134a: 0-10 |
| | | HFC-152a; 0-10 |
| 1-99 | (E,Z)-HFC-1225ye; 99-1 | HFC-134a: 0-10 |
| | (Molar ratio Z:E = 1:99 to | HFC-152a; 0-10 |
| | 99:1) | |
| 1-99 | (E)-HFC-1225ye; 99-1 | HFC-134a: 0-10 |
| | | HFC-152a; 0-10 |
| 1-98 | HFC-1225ye; 98-1 | Any*; 1 to 50 |
| 1-99 | Azeotrope-like | |
| | composition formed from | |
| | (Z)-HFC-1225ye and | |
| | HFC-1243yf; 99-1 | |
| 1-98 | HFC-1225ye; 98-1 | HFC-134a; 1-50 |
| 1-98 | Azeotrope-like | HFC-134a; 1-50 |
| | composition formed from | |
| | (Z)-HFC-1225ye and | |
| | HFC-1243yf; 98-1 | |
| 1-98 | HFC-1225ye; 98-1 | HFC-227ea; 1-30 |
| 1-98 | HFC-1225ye; 99-1 | HFC-152a; 1-50 |
| 1-98 | Azeotrope-like | HFC-125a; 1-50 |
| | composition formed from | |
| | (Z)-HFC-1225ye and | |
| | HFC-1243yf; 98-1 | |
| 1-98 | HFC-1225ye; 98-1 | CO₂; 0.1-50 |
| 1-97.9 | HFC-1225ye; 97.9-1 | HFC-134a; 1-50 |
| | | CO₂; 0.1-30 |
| 1-97.9 | HFC-1225ye; 97.9-1 | HFC-152a; 1-50 |
| | | CO₂; 0.1-30 |
| 1-97 | HFC-1225ye; 97-1 | HFC-134a; 1-30 |
| | | HFC-152a; 1-50 |
| | | CO₂; 0-30 |
| 1-97 | HFC-1225ye; 97.9-1 | HFC-227ea; 1-30 |
| | | CO₂; 0.1-30 |

TABLE 4
Compositions comprising CF₃—O—CF═CF₂ and HFC-1234ze

| | |
|---|---|
| Content of | Content of compound or |
| CF₃—O—CF═CF₂ | compounds B [% by weight] |

| [% by weight] | Fluoropropene | Other |
|---|---|---|
| 1-99 | HFC-1234ze; 99-1 | |
| 1-98 | HFC-1234ze; 98-1 | Any*; 1 to 50 |
| 1-98 | HFC-1234ze; 98-1 | HFC-134a; 1-50 |
| 1-98 | HFC-1234ze; 98-1 | HFC-227ea; 1-30 |
| 1-98 | HFC-1234ze; 99-1 | HFC-152a; 1-50 |
| 1-98 | HFC-1234ze; 98-1 | CO₂; 0.1-50 |
| 1-97.9 | HFC-1234ze; 97.9-1 | HFC-134a; 1-50 |
| | | CO₂; 0.1-30 |
| 1-97.9 | HFC-1234ze; 97.9-1 | HFC-152a; 1-50 |
| | | CO₂; 0.1-30 |
| 1-97 | HFC-1234ze; 97.9-1 | HFC-227ea; 1-30 |
| | | CO₂; 0.1-30 |
| 1-97 | HFC-1234ze; 97-1 | HFC-134a; 1-30 |
| | | HFC-152a; 1-50 |
| | | CO₂; 0-30 |

TABLE 5
Compositions comprising CF₃—O—CF═CF₂ and HFC-1234yf

| | |
|---|---|
| Content of | Content of compound or |
| CF₃—O—CF═CF₂ | compounds B [% by weight] |

| [% by weight] | Fluoropropene | Other |
|---|---|---|
| 1-99 | HFC-1234yf; 99-1 | |
| 1-98 | HFC-1234yf; 98-1 | Any*; 1 to 50 |
| 1-98 | HFC-1234yf; 98-1 | HFC-134a; 1-50 |
| 1-98 | HFC-1234yf; 98-1 | HFC-227ea; 1-30 |
| 1-98 | HFC-1234yf; 99-1 | HFC-152a; 1-50 |
| 1-98 | HFC-1234yf; 98-1 | CO₂; 0.1-50 |
| 1-97.9 | HFC-1234yf; 97.9-1 | HFC-134a; 1-50 |
| | | CO₂; 0.1-30 |
| 1-97.9 | HFC-1234yf; 97.9-1 | HFC-152a; 1-50 |
| | | CO₂; 0.1-30 |
| 1-97 | HFC-1234yf; 97.9-1 | HFC-227ea; 1-30 |
| | | CO₂; 0.1-30 |
| | | Preferred: |
| 1-97 | HFC-1234yf; 97-1 | HFC-134a; 1-30 |
| | | HFC-152a; 1-50 |
| | | CO₂; 0-30 |

TABLE 6
Compositions comprising CF₃—O—CF═CF₂ and HFC-1234ye

| | |
|---|---|
| Content of | Content of compound or |
| CF₃—O—CF═CF₂ | compounds B [% by weight] |

| [% by weight] | Fluoropropene | Other |
|---|---|---|
| 1-99 | HFC-1234ye; 99-1 | |
| 1-98 | HFC-1234ye; 98-1 | Any*; 1 to 50 |
| 1-98 | HFC-1234ye; 98-1 | HFC-134a; 1-50 |
| 1-98 | HFC-1234ye; 98-1 | HFC-227ea; 1-30 |
| 1-98 | HFC-1234ye; 99-1 | HFC-152a; 1-50 |
| 1-98 | HFC-1234ye; 98-1 | CO₂; 0.1-50 |
| 1-97.9 | HFC-1234ye; 97.9-1 | HFC-134a; 1-50 |
| | | CO₂; 0.1-30 |
| 1-97.9 | HFC-1234ye; 97.9-1 | HFC-152a; 1-50 |
| | | CO₂; 0.1-30 |
| 1-97 | HFC-1234ye; 97.9-1 | HFC-227ea; 1-30 |
| | | CO₂; 0.1-30 |
| 1-97 | HFC-1234ye; 97-1 | HFC-134a; 1-30 |
| | | HFC-152a; 1-50 |
| | | CO₂; 0-30 |

TABLE 7
Compositions comprising CF₃—O—CF═CF₂ and HFC-1243zf

| | |
|---|---|
| Content of | Content of compound or |
| CF₃—O—CF═CF₂ | compounds B [% by weight] |

| [% by weight] | Fluoropropene | Other |
|---|---|---|
| 1-99 | HFC-1243zf; 99-1 | |
| 1-98 | HFC-1243zf; 98-1 | Any*; 1 to 50 |
| 1-98 | HFC-1243zf; 98-1 | HFC-134a; 1-50 |
| 1-98 | HFC-1243zf; 98-1 | HFC-227ea; 1-30 |
| 1-98 | HFC-1243zf; 99-1 | HFC-152a; 1-50 |
| 1-98 | HFC-1243zf; 98-1 | CO₂; 0.1-50 |
| 1-97.9 | HFC-1243zf; 97.9-1 | HFC-134a; 1-50 |
| | | CO₂; 0.1-30 |
| 1-97.9 | HFC-1243zf; 97.9-1 | HFC-152a; 1-50 |
| | | CO₂; 0.1-30 |
| 1-97 | HFC-1243zf; 97.9-1 | HFC-227ea; 1-30 |
| | | CO₂; 0.1-30 |
| 1-97 | HFC-1243zf; 97-1 | HFC-134a; 1-30 |
| | | HFC-152a; 1-50 |
| | | CO₂; 0-30 |

In this embodiment, ternary compositions comprising CF₃—O—CF═CF₂, HFC-1225ye (including only the E isomer, only the Z isomer or mixtures of the E and Z isomer) and HFC-134a, ternary compositions comprising CF₃—O—CF═CF₂, HFC-1225ye (including only the E isomer, only the Z isomer or mixtures of the E and Z isomer) and HFC-152a and binary compositions consisting of CF₃—O—CF═CF₂, HFC-1225ye (including only the E isomer, only the Z isomer or mixtures of the E and Z isomer) wherein HFC-1225ye is comprised in an amount rendering the composition non-flammable are especially preferred.

CF₃—O—CF═CF₂ has a GWP around 0. The GWP of HFC-1225ye is assumed to be very low. Under the assumption that HFC-134a has a GWP of 1300, compositions comprising CF₃—O—CF═CF₂, HFC-1225ye and around 11.5% by weight of HFC-134a or less are calculated to have a GWP of less than 150. Compositions comprising around 10.7% by weight of 134a or less are calculated to have a GWP of less than 140. Compositions comprising 9.5% by weight of HFC-134a are calculated to have a GWP of less than 120 and are very preferred. Under the assumption that HFC-134a has a GWP of 1410, compositions comprising CF₃—O—CF═CF₂, HFC-1225ye and around 10.6% by weight of HFC-134a or less are calculated to have a GWP of less than 150. Compositions comprising around 10.0% by weight of 134a or less are calculated to have a GWP of less than 140. Compositions comprising 8.6% by weight of HFC-134a are calculated to have a GWP of less than 120. Hence, compositions of CF₃—O—CF═CF₂, HFC-1225ye and HFC-134a with a content equal to or less than 10.6% by weight, preferably equal to or less than 10.0% by weight, especially equal to or less than 8.6% by weight of HFC-134a are highly suitable. The molar ratio of CF₃—O—CF═CF₂ and HFC-1225ye is very flexible and can be selected between 1:99 and 99:1. Advantageously, it is selected thus that a low flammable or even non-flammable composition results.

HFC-152a is known to have a GWP of 140. Hence, compositions comprising CF₃—O—CF═CF₂ and HFC-1225ye with a content equal to or less than 85% by weight of HFC-152a are preferred, because they have a GWP of less than 120. It may be very advantageous to select the amounts of CF₃—O—CF═CF₂, HFC-1225ye and HFC-152a such that a low flammability is achieved or even non-flammability.

Ternary compositions of CF₃—O—CF═CF₂ and HFC-1225ye and HFC-134a, HFC-152a or HFC-227ea are very suitable as refrigerants. Examples for such ternary compositions are given in table 8. The respective components add up to 100% by weight.

TABLE 8
Ternary compositions of CF₃—O—CF═CF₂, the essentially pure
isomers or any mixtures of HFC-1225ye (for example those with an E/Z
ratio of 70:30 as available from ABCR; and 1:1 and 30:70 obtainable by
mixing the respective isomers) and HFC-134a or HFC-152a

| Content of | Content of compounds | |
|---|---|---|
| CF₃—O—CF═CF₂ | B [% by weight] | |
| [% by weight] | HFC-1225ye | Other |
| 79-83 | (Z)-HFC-1225ye: 9-11 | HFC-134a: 7-10 |
| 80 | (Z)-HFC-1225ye: 10 | HFC-134a: 10 |
| 82 | (Z)-HFC-1225ye: 10 | HFC-134a: 8 |
| 81-83 | (Z)-HFC-1225ye: 9-11 | HFC-134a: 7-8 |
| 79-83 | (E,Z)-HFC-1225ye: 9-11 | HFC-134a: 7-10 |
| | (Molar ratio Z:E = 1:99 to | |
| | 99:1) | |
| 80 | (E,Z)-HFC-1225ye: 10 | HFC-134a: 10 |
| | (Molar ratio Z:E = 1:99 to | |
| | 99:1) | |
| 81-83 | (E,Z)-HFC-1225ye: 9-11 | HFC-134a: 7-8 |
| | (Molar ratio Z:E = 1:99 to | |
| | 99:1) | |
| 82 | (E,Z)-HFC-1225ye: 10 | HFC-134a: 8 |
| | (Molar ratio Z:E = 1:99 to | |
| | 99:1) | |
| 79-83 | (E)-HFC-1225ye: 9-11 | HFC-134a: 7-10 |
| 80 | (Z)-HFC-1225ye: 10 | HFC-134a: 10 |
| 81-83 | (E)-HFC-1225ye: 9-11 | HFC-134a: 7-8 |
| 82 | (E)-HFC-1225ye; 10 | HFC-134a: 8 |
| 81-83 | (Z)-HFC-1225ye: 9-11 | HFC-152a: 7-8 |
| 82 | (Z)-HFC-1225ye: 10 | HFC-152a: 8 |
| 81-83 | (E,Z)-HFC-1225ye: 9-11 | HFC-152a: 7-8 |
| | (Molar ratio Z:E = 1:99 to | |
| | 99:1) | |
| 82 | (E,Z)-HFC-1225ye: 10 | HFC-152a: 8 |
| | (Molar ratio Z:E = 1:99 to | |
| | 99:1) | |
| 81-83 | (E)-HFC-1225ye: 9-11 | HFC-152a: 7-8 |
| 82 | (E)-HFC-1225ye: 10 | HFC-152a: 8 |

Binary compositions of CF₃—O—CF═CF₂ and HFC-1225ye (including only the E isomer, only the Z isomer or any mixtures of the E and Z isomer) are highly suitable.

Table 9 shows exemplary preferred compositions consisting of CF₃—O—CF═CF₂ and the essentially pure isomers and mixtures of HFC-1225ye. Hereby, the term “essentially pure isomer” preferably has the meaning that at most 10% by weight, preferably at most 5% by weight of the respective other isomer is contained.

TABLE 9
Binary compositions of CF₃—O—CF═CF₂ and the essentially pure
isomers and mixtures of HFC-1225ye (E/Z ratio of 70:30 as available
from ABCR; and 1:1 and 30:70 obtainable by mixing the respective
isomers)

| | | HFC-1225ye, Z |
|---|---|---|
| CF₃—O—CF═CF₂ [% by | HFC-1225ye, E isomer | isomer [% by |
| weight] | [% by weight] | weight] |
| 5 | 95 | |
| 5 | | 95 |
| 5 | 28 | 67 |
| 5 | 67 | 28 |
| 5 | 47.5 | 47.5 |
| 10 | 90 | |
| 10 | | 90 |
| 10 | 27 | 63 |
| 10 | 63 | 27 |
| 10 | 45 | 45 |
| 15 | 85 | |
| 15 | | 85 |
| 15 | 26 | 59 |
| 15 | 59 | 26 |
| 15 | 42.5 | 42.5 |
| 20 | 80 | |
| 20 | | 80 |
| 20 | 24 | 56 |
| 20 | 56 | 24 |
| 20 | 40 | 40 |
| 25 | 75 | |
| 25 | | 75 |
| 25 | 23 | 52 |
| 25 | 52 | 23 |
| 25 | 37.5 | 37.5 |
| 30 | 70 | |
| 30 | | 70 |
| 30 | 21 | 49 |
| 30 | 49 | 21 |
| 30 | 35 | 35 |
| 35 | 65 | |
| 35 | | 65 |
| 35 | 20 | 45 |
| 35 | 45 | 20 |
| 35 | 32.5 | 32.5 |
| 40 | 60 | |
| 40 | | 60 |
| 40 | 18 | 42 |
| 40 | 42 | 18 |
| 40 | 30 | 30 |
| 45 | 55 | |
| 45 | | 55 |
| 45 | 17 | 38 |
| 45 | 38 | 17 |
| 45 | 27.5 | 27.5 |
| 50 | 50 | |
| 50 | | 50 |
| 50 | 15 | 35 |
| 50 | 35 | 15 |
| 50 | 25 | 25 |
| 55 | 45 | |
| 55 | | 45 |
| 55 | 14 | 31 |
| 55 | 31 | 14 |
| 55 | 22.5 | 22.5 |
| 60 | 40 | |
| 60 | | 40 |
| 60 | 12 | 28 |
| 60 | 28 | 12 |
| 60 | 20 | 20 |
| 65 | 35 | |
| 65 | | 35 |
| 65 | 10 | 25 |
| 65 | 25 | 10 |
| 65 | 17.5 | 17.5 |
| 70 | 30 | |
| 70 | | 30 |
| 70 | 9 | 21 |
| 70 | 21 | 9 |
| 70 | 15 | 15 |
| 75 | 25 | |
| 75 | | 25 |
| 75 | 8 | 17 |
| 75 | 17 | 8 |
| 75 | 12.5 | 12.5 |
| 80 | 20 | |
| 80 | | 20 |
| 80 | 6 | 14 |
| 80 | 14 | 6 |
| 80 | 10 | 10 |
| 85 | 15 | |
| 85 | | 15 |
| 85 | 5 | 10 |
| 85 | 10 | 5 |
| 85 | 7.5 | 7.5 |
| 90 | 10 | |
| 90 | | 10 |
| 90 | 3 | 7 |
| 90 | 7 | 3 |
| 90 | 5 | 5 |
| 95 | 5 | |
| 95 | | 5 |
| 95 | 1 | 4 |
| 95 | 4 | 1 |
| 95 | 2.5 | 2.5 |

Also in this embodiment, the compositions of said compounds of formula (I) or (II) and unsaturated hydrofluorocarbons may additionally comprise compounds C, for example, stabilizers, for example, terpenes, compatibilizers, e.g. polyoxylkylene glycol ethers, amides, ketones, nitrites, chlorocarbons, fluoroethers, lactones or esters; UV fluorescent dyes, e.g. from the group of naphthalimides, perylenes, coumarins, anthracenes, phenantracenes, fluoresceins, xanthenes, thioxanthenes, naphthoxanthenes or their derivatives; lubricants, e.g. selected from mineral oils, PAG oils, alkylbenzenes, synthetic paraffins, synthetic naphthenes and poly(alpha)olefins; stabilizers, preferably terpenes, free radical scavengers, water scavengers, antioxidants, and tracer compounds for detection of dilution, contamination or other alteration of the composition.

The advantage of using compositions of compounds of formula (I), especially CF₃—O—CF═CF₂, and unsaturated hydrofluorocarbons, especially HFC-1225ye or the tetrafluoropropenes and optionally other compounds B, for example, HFC-134a and/or HFC-152a, is that they have reduced flammability or even are non-flammable, and reduced toxicity.

A very preferred composition comprises HFC-1225ye and CF₃—O—CF═CF₂ in a weight ratio of 90±2:10±2. An especially preferred composition consists of HFC-1225ye and CF₃—O—CF═CF₂ in a weight ratio of 90±2:10±2. Here, the constituents HFC-1225ye and CF₃—O—CF═CF₂ add up to 100% by weight. HFC-1225ye can be applied in the form of the (E) isomer, the (Z) isomer or any mixture of the (E) and (Z) isomers. As with the other compositions which comprise HFC-1225ye, also here the (Z) isomer of HFC-1225ye is the preferred isomer. Of course, for application as refrigerants or for other purposes, compounds C may be added, for example, stabilizers, compatibilizers or lubricants. Examples of compositions are compiled in the following table 10

TABLE 10
Preferred binary compositions of CF₃—O—CF═CF₂ and the essentially
pure isomers and mixtures of HFC-1225ye (E/Z ratio of 70:30 as
available from ABCR; and 1:1 and 30:70 obtainable by mixing the
respective isomers).

| CF₃—O—CF═CF₂ | |
|---|---|
| [% by weight] | HFC-1225ye [% by weight] |
| 88 | (E) isomer: 12 |
| 88 | (Z) isomer: 12 |
| 88 | (E) and (Z) isomer (in a 70:30 ratio of |
| | E/Z isomer): 12 |
| 90 | (E) isomer: 10 |
| 90 | (Z) isomer: 10 |
| 90 | (E) and (Z) isomer (in a 70:30 ratio of |
| | E/Z isomer): 10 |
| 92 | (E) isomer: 8 |
| 92 | (Z) isomer: 8 |
| 92 | (E) and (Z) isomer (in a 70:30 ratio of |
| | E/Z isomer): 8 |

These compositions, especially the compositions consisting of 90% by weight of HFC-1225ye and 10% by weight of CF₃—O—CF═CF₂, have surprising advantages. They are non-flammable; they have a very low temperature glide; they have a high coefficient of performance (Cop). They have an acceptable refrigerating capacity Qᵥₒₗ; the GWP is excellent, namely only approximately 1. The ODP is zero. Tests have shown that they have a good miscibility and compatibility with oils (lubricants), specially PAG oils, have a good material compatibility with metals and plastics which are used in refrigeration apparatus as o-rings, or hoses, a low permeability through plastics, a good stability, and a good thermodynamic behaviour. They also are miscible with terpenes, for example, α-pinene or β-pinene. Some data are given in the experimental section.

The method for heating or cooling using the compositions described above, especially the non-flammable compositions, is especially suitable for mobile cooling in cars, lorries for the driver's cabin or the goods to be transported, buses, trains, airplanes, ships, space ships or refrigerated transport boxes or refrigerated containers. Of course, it can also be applied in stationary machinery, for example, household appliances (freezers in private locations such as households) or industrial locations such as working rooms, production sites, hospitals, devices or rooms for storing or treating food or drugs, in heat pumps.

For many applications, for example mobile air conditioning, the apparatus often is designed to cool down to approximately 5° C. Here, the pressure at condensation often is about 12 to 18 bars (abs), preferably about 15 bars (abs.). The pressure at evaporation often is about 3 to 4 bars (abs.), preferably about 3.5 bars (abs.). The upper limit of the temperature of the room or item to be cooled is often about 55° C., while the temperature achievable by cooing is, as mentioned, about 5° C.

For freezers, the values can be somewhat different. Typical upper temperature limit of the room to be cooled is 55° C., the pressure of the refrigerant at condensation is around 15 bars (abs.). The temperature to be achieved by cooling is often −10° C., the pressure of the refrigerant at evaporation at that temperature is often around 1.5 bars (abs.).

For certain compositions, for example those comprising CF₃—O—CF═CF₂ and CF₃I, HFC-134a, HFC-152a and/or one or more of the tetrafluoropropenes and pentafluoropropenes, especially binary mixtures of CF₃—O—CF═CF₂ and the (E), isomer of HFC-1225ye, the (Z) isomer—which is especially preferred—or mixtures thereof, and optionally lubricants, stabilizers and/or other additives, it is possible to use them preferably as drop-in or retrofit for machines which are designed to operate with HFC-134a or similar refrigerants. “Drop-in” means the lubrication oil can be reused, while “retrofit” means that suitable fresh oil must be used. For machines which are designed to operate with refrigerants with higher or lower boiling point than HFC-134a, it is possible to tailor compositions with a boiling point comparable to the boiling point of the refrigerant to be substituted by selecting suitable compounds A and/or B. For example, to the compositions mentioned above; especially those comprising CF₃—O—CF═CF₂ and CF₃I, HFC-134a, HFC-152a or one or more of the tetrafluoropropenes and pentafluoropropenes, one can add a compound A and/or B with higher or lower boiling point, respectively, than the boiling point of CF₃—O—CF═CF₂ and CF₃I, HFC-134a, HFC-152a or one or more of the tetrafluoropropenes and pentafluoropropenes. Such tailored compositions can be used as a drop-in or retrofit for refrigeration machines designed to operate with refrigerants of higher or lower boiling point than HFC-134a. For example, C₂F₅—O—CF═CF₂ might be a substitute for R11.

It is advantageous that the refrigerant compositions of the present invention are suitable for heat exchangers manufactured at least partially from aluminium parts which are brazed using non-corrosive fluxes, especially alkali fluoroaluminates such as potassium fluoroaluminate or cesium-containing potassium fluoroaluminate, or potassium hexafluorosilicate.

Another aspect of the present invention are compositions of matter, which are suitable for performing the methods for heating and cooling as described above, but also for many other purposes. According to this aspect of the present invention, the composition of matter in the gaseous or liquid state comprises at least one compound of formula (I) or formula (II) and at least one other chemical compound. The term “other chemical compound” in the claimed composition of matter does not include unwanted impurities. Unwanted impurities are for example residual starting compounds, compounds which are the result of side reactions, or, for example, substances which are comprised in air. Preferred chemical compounds are those selected from compounds A provided that the compound of formula (I) or (II) and the other chemical compound A are different. Preferred chemical compounds can also be selected from compounds B and compounds C. Suitable compounds B and C and preferred compounds B and C are described above. Accordingly, the refrigerant compositions disclosed herein before which correspond to this definition are preferred compositions of matter according to the invention.

Of course, the composition of matter can also comprise two or more compounds B and/or compounds C. Certain of these compositions may be azeotropic or quasi-azeotropic. It is expected that mixtures of CF₃—O—CF═CF₂ and CF₃I are quasi-azeotropic.

Preferred compositions of matter suitable as refrigerant are those wherein the at least one other chemical compound is selected from compounds B denoting the group consisting of flammable liquids or gases or non-flammable liquids or gases, or compounds C denoting additives, preferably lubricants, stabilizers, metal passivators, and corrosion inhibitors.

Preferred compositions of matter suitable as refrigerant correspond to those preferred embodiments which have been mentioned above as preferred refrigerants to be applied in the method of the present invention. Often, they are those which comprise at least one compound of formula (I) or formula (II) and at least one non-flammable compound B, preferably selected from the group consisting of CF₃I, perfluorocarbons, saturated hydrocarbons, for example, HFC-32, HFC-134a, HFC-134, HFC-152a, the pentafluoropropenes, hexafluoropropanes and heptafluoropropanes, unsaturated hydrofluorocarbons, for example trifluoropropenes, tetrafluoropropenes, pentafluoropropenes, fluorinated ketones, for example, perfluoro-(methyl-isopropyl ketone), perfluoro-(ethyl-isopropyl ketone), saturated fluoroethers, for example trifluoromethyl-difluoromethylether (E-125), trifluoromethyl-fluoromethylether (E-134a), trifluoromethyl-methylether (E-143a), and carbon dioxide.

Very advantageous are compositions of matter suitable as refrigerant wherein the at least one non-flammable compound B is comprised in an amount effective to render the composition of matter non-flammable.

The composition of matter suitable as refrigerant may further comprise at least one compound C selected from the group consisting of lubricants, UV fluorescent dyes, tracer compounds, compatibilizers, stabilizers, metal passivators, and corrosion inhibitors.

Highly preferred compositions of matter suitable as refrigerant are those wherein CF₃—O—CF═CF₂ is comprised as compound of formula (I), and wherein the non-flammable compound B is selected from CF₃I, HFC-134, HFC-134a, HFC-152a, HFC-125, fluorinated propenes, especially trifluoropropenes, tetrafluoropropenes, pentafluoropropenes, HFC-227ea and CO₂.

According to one embodiment, compositions of matter suitable as refrigerant which comprise or consist of CF₃—O—CF═CF₂ as compound of formula (I), CF₃I and/or CO₂ as non-flammable compound B and optionally at least one compound C selected from the group consisting of a stabilizer, a lubricant, a metal passivator and a corrosion inhibitor are preferred.

According to another particularly preferred embodiment, compositions of matter comprise or consist of CF₃—O—CF═CF₂ and one or more of a saturated or unsaturated hydrofluorocarbon selected from the group consisting of HFC-134a, HFC-152a, the trifluoropropenes, the tetrafluoropropenes, and especially the pentafluorpropenes. Binary compositions of CF₃—O—CF═CF₂ and the (E) isomer of HFC-1225ye, the (Z) isomer or mixtures thereof are highly preferred; especially those with the (Z) isomer. Ranges are given above. A mixture of 90±2% by weight CF₃—O—CF═CF₂ and 10±2% by weight of one of the isomes or mixtures thereof of HFC-1225ye are highly preferred. Ternary compositions of CF₃—O—CF═CF₂, HFC-134a and the (E) isomer of HFC-1225ye, the (Z) isomer or mixtures thereof are also very suitable; especially those with the (Z) isomer.

The compositions according to the present invention are, for example, suitable for heating and cooling, thus constituting a refrigerant, as described above. Such compositions may comprise or essentially consist of one or more of compounds A and one or more of compounds B, of one or more of compounds A and one or more of compounds C, or of one or more of compounds A, one or more of compounds B and one or more of compounds C. Such compositions which are suitable as refrigerant are described above in detail.

The compositions of matter can be used for many other purposes. For example, they can be used as heat transformer liquids, as liquid in heat pipe applications, ORC processes, heat transfer applications and solvent applications, or they can be used as foam blowing agent for polyurethane foams or thermoplastic foams, for example polystyrene foams. They may be used as solvents, as cleaning agents or as fire-extinguishing agent. They also may be used for preparing aerosols. Also in such fields of application, they may additionally comprise auxiliaries useful for that purpose. Examples of auxiliaries are stabilizers, dyes, and catalysts.

The compounds of formulae (I) and (II) often have good thermal stability properties. For example, substantially no degradation can be observed when keeping the preferred compound of formula (I), CF₃—O—CF═CF₂ two weeks at a temperature of 150° C. without any degradation. This temperature is far above even extreme temperatures occurring in compressors: most often, the temperature in compressors does not exceed 120° C. Accordingly, refrigerants containing compounds of formulae (I) and (II) and in particular CF₃—O—CF═CF₂ display particularly adequate thermal stability properties. The compounds of formulae (I) and (II) and in particular CF₃—O—CF═CF₂ can consequently be used to prevent or reduce thermal decomposition or to improve thermal stability of refrigerants, preferably of mobile air conditioning (MAC) systems.

For some of these purposes, e.g. for heat pipe applications, ORC processes, heat transfer applications and solvent applications, compositions comprising compounds of formula (I) or (II) and/or compounds B with a boiling point in the range of 20 to 70° C. are especially suitable.

Another object of the present invention is the use of the compounds of formulae (I) and (II) and of the compositions of matter comprising or consisting essentially of compounds of formula (I) or (II) as refrigerants, heat transformer liquids, for heat pipe applications, ORC processes, heat transfer applications and solvent applications, blowing agents for foam preparation, in aerosol generating fluids and fire extinguishants, or as part of refrigerants. For some of these purposes, e.g. for heat pipe applications, ORC processes, heat transfer applications and solvent applications, the use of compounds of formula (I) or (II) with a boiling point in the range of 20 to 70° C. or of compositions, comprising them, with a boiling point in that range is especially advantageous.

Preferably, a non-flammable gas is co-used in an amount which renders the compounds of formula (I) or (II) non-flammable. If the use pertains to the application as solvent, compound(s) of formulae (I) or (II) and compounds B with a boiling point in the range of 20 to 70° C. are used preferably.

The term “other chemical compound” denotes in a preferred embodiment chemical compounds which are gaseous at standard conditions (1 bar abs, 25° C.) or liquid and support the compounds of formulae (I) or (II) in the intended purpose. For example, if the composition of matter is intended to be used as a refrigerant, the “at least one other chemical compound” can be a compound known as a constituent of refrigerants, e.g. a refrigerating agent, a stabilizer, a lubricant or other additive as described above. If the composition of matter of the present invention is applied as blowing agent for foams, the “at least one other component” may be a known blowing agent or a catalyst used for that purpose. If the intended purpose is to provide a fire extinguishing agent, the “at least one other chemical compound” may be a known fire extinguishant or auxiliaries present in extinguishing compositions. Examples are HCFs 23, 134a, 227ea, 245fa, 236ea, perfluoro-ethylisopropylketone or a propellant such as nitrogen, the propellant preferably being present in an amount to provide a pressure of up to 20 bars abs. If the composition of matter is to be used as a solvent, the “at least one other chemical compound” may for example be a known solvent.

The composition of matter preferably has a GWP lower than 150, more preferably lower than 140, especially preferably lower than 120.

The compositions of matter have a low acute toxicity and a low GWP. Preferred compositions of matter have the inherent property of being non-flammable.

Still another object of the present invention concerns systems comprising the composition of matter. Many embodiments of the composition of matter, and all embodiments where the preferred compound CF₃—O—CF═F₂ is comprised, contain one or more gaseous compounds. Hence, such composition of matter must be protected against evaporation into the air. Simple “systems” of the present invention are containers, mostly made of metal, which comprise the composition, e.g. pressure bottles. Preferred “systems” denote apparatus which allow the application of the composition of matter. For example, the system of the present invention may be a portable fire extinguisher or a total flooding system comprising the composition of matter.

A preferred system of the present invention is a machine for cooling or heating comprising the composition of matter according to the present invention. Usually, such a machine comprises a condenser, an evaporator, lines to transport the composition between the different parts of the apparatus, heat exchangers, valves, pumps and other parts used in such apparatus and can be used in a mobile or stationary way. Stationary systems are freezers, air conditioning systems in houses, factories, hospitals, working rooms, rooms for storing food or drugs, refrigerated boxes or containers, example for storage or transport.

A very preferred system of the present invention is a mobile air conditioning system, especially for cars, lorries, trucks, buses, airplanes, trains, spaceships and other mobile items.

The compounds of formula (I) can for example be prepared by the addition of perfluoroxyfluorides to 1,2-dichloro-1,2-difluoroethylene or 1,2-dibromo-1,2-difluoroethylene and subsequent reduction with Zinc. The first step is described in U.S. Pat. No. 4,900,872, the complete sequence is described by W. S. Durell et al. J. Polymer Sci. Part A, 3 (1965), pages 4065ff.

Trifluoropropenes, tetrafluoropropenes and pentafluoropropenes can exist as isomers. The term “trifluoropropenes” includes all isomers. An especially preferred compound among the trifluoropropenes is HFC-1243zf, which is 3,3,3-trifluoropropene. The term “tetrafluoropropenes” includes all possible isomers, especially HFC-1234ze, which 1,3,3,3-tetrafluoropropene, HFC-1234yf, which is 2,3,3,3-tetrafluoropropene, and HFC-1234ye, which is 1,2,3,3-tetrafluoropropene. The term “pentafluoropropenes” includes all isomers, e.g., 1,1,3,3,3-pentafluoropropene and especially HFC-1225ye, which is 1,2,3,3,3-pentafluoropropene, because it is non-flammable. The preparation of 3,3,3-trifluoropropene is, for example, described in U.S. Pat. No. 2,889,379, U.S. Pat. No. 4,465,786 and U.S. Pat. No. 4,798,818. Published US patent application 2005/0090698 discloses the preparation of certain trifluoropropenes, tetrafluoropropenes and pentafluoropropenes all of which have a trifluoromethyl group. EP-A-0 974571 discloses the preparation of 1,3,3,3-tetrafluoropropene. WO/1998/037043 discloses the preparation of 1,1,3,3,3-pentafluoropropene. Pentafluoropropenes are also described in U.S. Pat. No. 6,548,720 (as being precursors of saturated hydrofluorocarbons). 1,2,3,3,3-pentafluoropropene is available from SynQuest Laboratories, Inc., Alachua, Fla. 32616-0309; it is also available from ABCR GmbH & Co. KG, Karlsruhe/Germany (E:Z ratio 30:70). The trans-isomer (which is also denoted as E-isomer) of 1,2,3,3,3-pentafluoropropene can be prepared, for example, as described in the dissertation of Anwar Abo-Amer, An innovative method to generate Iodine (V and III)-Fluorine Bonds . . . , 2005, page 123, from hexafluoropropene, tri-n-butylphosphine and water in triglyme. U.S. Pat. No. 5,532,419 describes the preparation of trifluorobutene compounds. Isomer mixtures can also be prepared from 1,1,1,2,3,3-hexafluoropropane and bases like KCl, see D. Sianesi and R. Fontanelli, Ann. Chim. (Rome); 55; 1965; 850 to 861.

D. J. Burton et al. describe in J. Fluorine Chem., 44 (1989), pages 167 to 174 the preparation of the (E) and (Z) isomers of HFC-1225ye. The (E) isomer can be prepared by reaction of hexafluoropropene and tributylphosphine in ether and subsequent hydrolysis with water in triglyme. The (Z) isomer can be prepared from the (E) isomer by reacting it with SbF₅ at low temperature or photochemically.

If desired, compounds of formulae (I) and/or (II) can be purified before applying them as refrigerant or other purposes. For example, acidic constituents can be removed, e.g. by applying molecular sieve or other adsorbents for removing acidic matter.

A preferred refrigeration system according to the present invention is a system which is designed to operate with HFC-134a or hydrocarbons, and which is filled with a compound of formula (I) and/or (II) or with a composition comprising a compound of formula (I) and/or (II) and which comprises fresh lubricant (retrofit system) or used lubricant (drop-in system).

Another preferred refrigeration system according to the present invention is one which is designed to operate with HFC-134a and is adapted to operate with a refrigerant comprising at least one compound of formula (I) or (II) or the composition of matter suitable as refrigerant described above.

In the fore-going description of the different aspects of the present invention, a method of refrigeration, a composition of matter, the use of that composition of matter for several purposes and certain refrigeration systems are described which, in particularly preferred embodiments, operate with CF₃—O—CF═F₂ as compound of formula (I). According to another preferred embodiment, CF₃—O—CF═F₂ is to be substituted by C₃F₇—O—CF═CF₂ as compound of formula (I). To understand the details of this embodiment, in the fore-going description, the term “CF₃—O—CF═F₂” simply must be substituted by “C₃F₇—O—CF═CF₂”. Hence, methods of refrigeration performed with compositions of matter comprising C₃F₇—O—CF═CF₂ instead of CF₃—O—CF═F₂, the described uses of that composition or of CF₃—O—CF═F₂ as such, performed with C₃F₇—O—CF═CF₂ instead of CF₃—O—CF═F₂, and the described refrigeration system performed with a refrigerant comprising or consisting of C₃F₇—O—CF═CF₂ instead of CF₃—O—CF═F₂, are also embodiments of the present invention. C₃F₇—O—CF═CF₂ stands for i-C₃F₇—O—CF═CF₂ and n-C₃F₇—O—CF═CF₂.

Of course, when using C₃F₇—O—CF═CF₂ instead of CF₃—O—CF═F₂, slight modifications may be needed because of the different boiling point.

The following examples shall describe the invention in further detail without being intended to limit the scope of it.

### EXAMPLES

General remark. As a lubricant in refrigeration applications, e.g. for Mobile Air Conditioning, alkyl benzene lubricants with a kinematic viscosity at 40° C. in the range of 46 mm²/s, for example, Fuchs Reniso S46F, are very suitable. Instead of this oil, ND8 PAG of Denso can be applied.

### Example 1

### Preparation of a Composition of Matter Suitable for Refrigeration

1a) Preparation of a binary mixture: CF₃—O—CF═CF₂ and CF₃I are mixed so that a composition comprising 50% by weight of each of the components results. The mixture is filled into a pressurized bottle.
1b) Preparation of a refrigerant composition comprising the binary mixture of 1a):
   100 parts of the binary mixture of 1a), 20 parts of a polyolester lubricant and
   10 parts of an epoxide stabilizer are mixed under pressure to form a refrigerant composition which can be used in a mobile air conditioning system.
1c) Preparation of a refrigerant composition comprising the binary mixture of 1a):
   100 parts of the binary mixture of 1a), 20 parts of an alkyl benzene lubricant, e.g. Fuchs Reniso S46F, and 10 parts of an epoxide stabilizer are mixed under pressure to form a refrigerant composition which can be used in a mobile air conditioning system.

### Example 2

### A Mobile Air Conditioning System Comprising the Refrigerant of Example 1a)

Into a mobile air conditioning system, the used refrigerant (which may be, for example, HFC-134a) is removed, and 850 g of the refrigerant mixture of example 1a is filled into the system under pressure. The oil remains in the system. The air conditioning system is ready for use.

### Example 3

### Binary Mixture with 60% by Weight of CF₃—O—CF═CF₂

Example 1 is repeated. CF₃—O—CF═CF₂ is added in such an amount that the binary mixture with CF₃I consists of 60% by weight of CF₃—O—CF═CF₂ and 40% by weight CF₃I. After adding lubricant and stabilizer as described in example 2a), the refrigerant is ready for use in a mobile or stationary air conditioning system.

### Example 4

### Use of the Refrigerant of Example 3 as Drop-in for HFC-134a

A refrigeration system operated with HFC-134a is provided. The contained HFC-134a is removed from the system by applying a vacuum so that the system is essentially free of residual HFC-134a. The oil remains in the system for re-use. The refrigerant of example 3 is filled into the system which then can be brought into operation.

### Example 5

### Azeotropes from CF₃—O—CF═CF₂ and HFC-134a

The following compositions are produced by mixing CF₃—O—CF═CF₂ and HFC-134a:

| CF₃—O—CF═CF₂ | HFC-134a |
|---|---|
| 25 | 75 |
| 30 | 70 |
| 40 | 60 |
| 50 | 50 |
| 60 | 40 |

These mixtures form an azeotrope with a pressure of around 3.57 bars (absolute) at 0° C. (i.e., they have a lower boiling point than each of the both constituents).

### Example 6

### Thermal Stability Test of CF₃—OCF═CF₂

A sample of CF₃—O—CF═CF₂ was kept for 2 weeks at a temperature of 150° C. No decomposition was observed.

### Example 7

### Mixtures of CF₃—O—CF═CF₂ and HFC-134a

The following compositions are produced by mixing CF₃—O—CF═CF₂ and HFC-134a:

| CF₃—O—CF═CF₂ | HFC-134a |
|---|---|
| 89 | 11 |
| 90 | 10 |
| 90.5 | 9.5 |
| 91 | 9 |
| 91.5 | 8.5 |
| 92 | 8 |
| 93 | 7 |

These mixtures are very suitable as refrigerant and have low GWP values. The mixtures with 10 or less % by weight of HFC-134a, for example, have a GWP lower than 140. The mixtures with 9.5 or less, especially the mixtures with 8.5% by weight or less of HFC-134a have a GWP of less than 120.

### Example 8

### Mixtures of CF₃—O—CF═CF₂ and (Z)-HFC-1225ye, (E)-HFC-1225ye and Mixtures of the (Z) and (E) Isomer

8.1. Preparation of (Z)-HFC-1225ye, (E)-HFC-1225ye and mixtures thereof

8.1.1 (E)-1,2,3,3,3-pentafluoropropene [(E)-HFC-1225ye] can be prepared as described by D. J. Burton et al. in J. Fluorine Chemistry, 44 (1989), pages 167-174 by the reaction of hexafluoropropene and tri-n-butylphosphine in ether at −78° C., letting the reaction mixture warming up to room temperature and subsequent hydrolysis in triglyme by adding slowly and incrementally water. The (E)-isomer formed is transferred to a tube for storage by evaporation and subsequent condensation. Its boiling point is −18° C., see D. Sianesi and R. Fontanelli in Ann. Chim. (Rome); 55 (1965), pages 850 to 861, especially pages 853, 858 and 859.

8.1.2. (Z)-1,2,3,3,3-pentafluoropropene [(Z)-HFC-1225ye] can be produced from the (E)-isomer obtainable as described above by cooling SbF₅ in a reactor to the temperature of liquid nitrogen and slowly condensing the (E)-isomer to the cooled SbF₅. Per 30 g of the (E)-isomer, 3 to 4 ml of the antimony compound may be applied. The mixture is then allowed to warm to room temperature, the (Z)-isomer formed is transferred to a tube and contacted with NaF, and then transferred to storage. Its boiling point is −18.5° C., see D. Sianesi, R. Fontanelli, loc. cit. The transformation can also be performed by photochemical treatment.

8.1.3. Mixtures of (E)-Isomer and (Z)-Isomer

Such mixtures are obtainable commercially; for example, from ABCR GmbH & Co. KG, Karlsruhe/Germany, mixtures with an E:Z ratio of 70:30 are available.

Alternatively, mixtures may be produced as described in U.S. Pat. No. 5,679,875 by contacting 1,1,1,2,3,3-hexafluoro-propane at 430° C. with activated carbon, or as described in U.S. Pat. No. 6,031,141 by dehydrofluorination of 1,1,1,2,3,3-hexafluoropropane over chromium trifluoride at 350 to 400° C. Alternatively, they can be prepared in definite molar ratio by mixing appropriate amounts of the (E) and (Z) isomer which may be obtained as described above.

8.2. Preparation of the Mixtures

The binary and ternary mixtures are prepared by condensing the ether, the propene and, if applicable, the third component into a pressurized storage tank.

| CF₃—O—CF═CF₂ | | HFC-134a |
|---|---|---|
| | (E)-HFC-1225ye | |
| 90 | 10 | |
| 70 | 30 | |
| 60 | 40 | |
| 50 | 50 | |
| 40 | 60 | |
| 41 | 50 | 9 |
| 35 | 65 | |
| 30 | 70 | |
| 30 | 62 | 8 |
| 25 | 75 | |
| 20 | 80 | |
| 15 | 85 | |
| 10 | 82 | 8 |
| | (Z)-HFC-1225ye | |
| 90 | 10 | |
| 70 | 30 | |
| 60 | 40 | |
| 50 | 50 | |
| 41 | 50 | 9 |
| 40 | 60 | |
| 35 | 65 | |
| 30 | 70 | |
| 30 | 62 | 8 |
| 25 | 75 | |
| 20 | 80 | |
| 15 | 85 | |
| 10 | 82 | 8 |
| | (E,Z)-HFC-1225ye | |
| | (molar ratio E:Z = | |
| | 70:30) | |
| 90 | 10 | |
| 70 | 30 | |
| 60 | 40 | |
| 50 | 50 | |
| 40 | 60 | |
| 41 | 50 | 9 |
| 35 | 65 | |
| 30 | 70 | |
| 30 | 62 | 8 |
| 25 | 75 | |
| 20 | 80 | |
| 15 | 85 | |
| 10 | 82 | 8 |

### Example 15

### Application of Mixtures Comprising Pentafluoropropenes as Refrigerant

The mixtures described in example 8 are transferred from the pressurized storage tank in liquid form to the storage tank of a mobile air conditioning unit. Alkyl benzene lubricant, e.g. Fuchs S46F, can be added.

### Example 10

### 20/80 Mixture of Perfluoro-Methylvinyl Ether (PVME) and Pentafluoropropene

By condensing perfluoro-methylvinyl ether and HFC-1225ye mixtures with an E:Z ratio of 70:30, obtained from ABCR GmbH & Co. KG, Karlsruhe/Germany, a refrigerant composition with a weight ratio of PVME and pentafluoropropene of 20:80 was prepared. The composition was identified to be non-flammable.

### Example 11

### 10/90 Mixture of Perfluoromethyl-Vinyl Ether (PVME) and (E)/(Z)-Pentafluoropropene

By condensing perfluoro-methylvinyl ether and HFC-1225ye mixtures with an E:Z ratio of 70:30, obtained from ABCR GmbH & Co. KG, Karlsruhe/Germany, a refrigerant mixture with a weight ratio of PVME and pentafluoropropene of 10:90 was prepared.

The composition was identified to be non-flammable. With this mixture, tests concerning mechanical impact on materials used in refrigeration apparatus were performed, e.g. permeability etc, see below.

### Example 12

### 10/90 Mixture of Perfluoromethyl-Vinyl Ether (PVME) and (Z)-Pentafluoropropene

By condensing perfluoro-methylvinyl ether and (Z)-HFC-1225ye, a refrigerant composition with a weight ratio of PVME and (Z)-pentafluoropropene of 10:90 is prepared.

The mixture is identified to be non-flammable.

### Example 13

### 10/10/80 Mixture of Perfluoromethyl-Vinyl Ether (PVME), HFC-134a and Pentafluoropropene

By condensing perfluoro-methylvinyl ether, HFC-134a and HFC-1225ye, a refrigerant composition with a weight ratio of PVME, HFC-134a and pentafluoropropene of 10:10:80 is prepared.

The mixture is identified to be non-flammable.

### Example 14

### Thermodynamic Data of Some Compositions According to the Invention Compared to the Data of Pure HFC-134a

The thermodynamic data of the components perfluoromethyl-vinyl ether (PVME) and the (E)/(Z) mixture of pentafluoropropene used for example 11 were measured, and with these data, COP (coefficient of performance) and Q_{vol.} (volumetric efficiency) of the compositions of examples were calculated using “Refprop 7.0” for a single cycle with Tₒ=0° C., T_{subH.}=10 K, T_{subC}=2 K, T_{c}=40° C., η_{is.}=f(p_{c}/p₀).

The resulting figures were compared to those of HFC-134a.

| | | Composition of | Composition of |
|---|---|---|---|
| Property | HFC-134a* | example 11** | example 13*** |
| T Glide 0° C. - | 0 | 0.6 | 1.4 |
| T″ (p′(0° C.)), K | | | |
| Cₒₚ | 4.43 | 4.33 | 4.23 |
| Qᵥₒₗ | 2115 | 1471 | 1540 |

*Boiling point of the saturated liquid at 1.013 bar: −26.7° C.
**Boiling point of the saturated liquid at 1.013 bar: −18.4° C.
***Boiling point of the saturated liquid at 1.013 bar: −20.5° C.

The results show that the composition of example 11 performs very good and even better than the composition of example 13 because the higher the cop, the better the performance of the respective refrigerant.

The thermodynamic data for a composition of PVME and the (Z) isomer of HFC-1225ye, a preferred mixture, are comparable to those of compositions with the (E) and (Z) isomer mixture. The advantage is that the (Z) isomer is very stable.

### Example 16

### Mixtures Stabilized with Terpenes

To the mixture of example 11, β-pinene was added so that the content in β-pinene was 0.5% by weight of the composition.

Example 16 can be repeated with other stabilizers, for example other terpene compounds, for example, limonene, α-pinene, dipentene, or citronellol.

Determination of certain properties of the composition of example 11

### A) Permeability of Standard Sealing Materials:

General procedure: The tested polymers were applied as slabs with a thickness of 2 mm. They were put into a high pressure permeation cell. The refrigerant was filled into a space on one side of the slab. Permeated refrigerant was analyzed by gas chromatograph. The permeation cells were stored for 100 h at 90° C.

Slabs made of several EPDM (ethylene propylene diene monomer rubber) plastic, and HNBR (hydrogenated acrylnitrile butadiene rubber) plastic were tested.

The lowest permeation was observed for EPDM plastic slab made from material available from Freudenberg.

### B) Permeation of Hoses

General procedure: the refrigerant was filled into the respective hose. Then the hose was kept for 500 h at 90° C. The permeation was analyzed by gas chromatography of the gas space around the hose.

Several high temperature hoses and normal temperature hoses made from polyamide, chloroprene, butyl and rubber were tested. The permeation through hoses made from polyamide and chloroprene was very low.

The permeation tests also revealed that the rate of permeation of PVME through the different items is by far lower than the speed of permeation of the (E) and (Z) isomers of HFC-1225ye.

C) Compatibility of a Refrigerant/Oil Composition with Sealing, O-Ring and Slab Material

Several o-rings made of EPDM and HNBR material, several high temperature hoses made of polyamide and chloroprene and normal temperature hoses made of polyamide, butyl or comprising an inner butyl layer were tested.

Mechanical properties (tensile properties, IRHD (international rubber hardness degree), geometrical dimensions and hardness) were determined.

General Procedure:

The tests were performed in autoclaves suitable for the o-rings and dumb bells, respectively. The hose material was applied to dumb bells. 60 ml of ND8 PAG oil and the material to be tested was put into the autoclave which then was evacuated were evacuated, and 60 ml refrigerant (for oil rings) or 30 ml (for dumb bells) were condensed in the autoclave. The autoclave was then transferred to a thermo chamber and, in case of the o-rings, kept there for 500 h at 100° C. and, in another experiment, for 168 h at 150° C. The high temperature hose material was tested for 500 h at 100° C. and for 168 h at 140° C. The normal temperature hose material was treated for 500 h at 100° C. and 168 h at 125° C. The refrigerant was then removed, the samples were pruned to remove adhering oil, afterwards, the samples were kept for 30 minutes at 60° C. in a thermo chamber.

Tensile properties were then tested according to ISO 37 (2005), IRDH hardness according to ISO 48 (2003). Dimensions and volumes were determined by measuring the dimensions and calculation of the volumes and their changes.

Results:

Changes of volume: target limit±15%. All tested samples fulfilled this requirement.

Change of elongation: target limit: ±50%. All treated samples fulfilled this condition.

Change of tensile strength: target limit: ±30%. All o-rings (made of EPDM and HNBR) and normal temperature hose material (made of polyamide and butyl) fulfilled the conditions, as well as high temperature hose made of CR-PA-NBR-PVA-CIIR) and one of those made of polyamide (Goodyear 4890). A chloroprene and another polyamide hose material was slightly out of the target range. A hose material comprising only a chloroprene inner tube layer was out of the range.

Change of hardness (IRHD-M): target range is ±15%. All o-rings, all high temperature and normal temperature hose material fulfilled the condition.

Compatibility with metals: The tests were performed according to ASHRAE standard 97. 1 g of lubricant, 1 g of refrigerant and 0.5% by weight, based on the total weight of the mixture, of β-pinene as stabilizer. Copper, steel or aluminium chips were used. A specified moisture content was adjusted. The tests were performed in a sealed tube. Then, the liquid and the metal chips were evaluated.

Treatment 190° C./24 h, moisture content 201 ppm: the metal chips remained unchanged, the color of the liquid turned a little darker (color=3.0 versus 2.0 of the unaged liquid).

Treatment 190° C./24 h, moisture content 3983 ppm: the metal chips remained unchanged, the liquid turned a little darker (2.5 for the aged liquid, versus 2.0 for the unaged liquid).

Treatment 175° C./14 days, moisture content 201 ppm: the metal chips remained unchanged, the color of the liquid turned a little darker (color=3.5 versus 2.0 of the unaged liquid).

Treatment 175° C./14 days, moisture content 3983 ppm: the metal chips remained unchanged, the liquid turned a little darker (3.0 for the aged liquid, versus 2.0 for the unaged liquid).

The analytical result of the unaged liquid revealed that the samples with higher moisture content were in fact more stable than the samples with low moisture content.

Oil miscibility: The oil miscibility was measured using Fuchs Reniso S46F with lubricant concentrations of 4, 7, 10, 20, 30 and 50% by weight. At 22-23° C. and at cooling to −40° C., there remained one clear phase. Upon heating to 100° C., the sample with 50% by weight of the lubricant remained one phase. The other samples formed two phases: 4% by weight of oil at 85° C., 7% by weight of oil at 82° C., 10% by weight of oil at 91° C., 20% by weight of oil at 82° C., 30% by weight of oil at 93° C.

1. A method for heating or cooling with a refrigerant comprising one or more compounds A of the general formula (I)
CₓF_{y}—O—CF═CFW (I)
wherein x is 1, 2, 3, 4, 5 or 6 and y=2x+1, and wherein W is F, CF₃, C₂F₅, or C₃F₇ or of general formula (II)
CₓF_{y}—O—CX═CYZ (II)
wherein x and y have the meaning given above, X stands for H or F, Y stands for H or F and Z stands for H, F, CF₃ or C₂F₅ with the proviso that at least one of X, Y or Z are H, and not more than 2 hydrogen atoms are contained in the compound of general formula (II).

2. The method according to claim 1, wherein the refrigerant further comprises at least one compound B which has refrigerant properties, and/or at least one compound C which is an refrigerant additive.

3. The method according to claim 1, wherein the refrigerant comprises at least one non-flammable compound B which reduces the flammability of the refrigerant.

4. The method according to claim 3, wherein the non-flammable compound B is selected from the group consisting of CF₃I, perfluorocarbons, saturated or unsaturated hydrofluorocarbons, fluorinated ketones, saturated fluoroethers, and carbon dioxide.

5. The method according to claim 4, wherein the non-flammable compound B is selected from the group consisting of C1 to C6 hydrofluorocarbons, pentafluoropropanes, hexafluoropropanes, heptafluoropropanes, tetrafluoropropenes, pentafluoropropenes, and CO₂.

6. The method according to claim 1, being suitable for mobile air cooling.

7. The method according to claim 1, wherein the refrigerant is a drop-in or retrofit substitute for HFC-134a.

8. The method according to claim 1, wherein the refrigerant comprises at least one additives as compounds C, said additive being selected from the group consisting of lubricants, stabilizers, metal passivators, corrosion inhibitors, tracer compounds, oil compatibilizers, and UV fluorescent dyes.

9. The method according to claim 8, wherein the refrigerant comprises at least one stabilizer.

10. The method according to claim 1, wherein the refrigerant comprises or consists of CF₃—O—CF═CF₂ and at least one compound selected from the group consisting of CF₃I; HFC-134a; a pentafluoropropene selected from CF₃—CH═CF₂, (E)-CF₃—CF═CHF, (Z)-CF₃—CF═CHF, or mixtures of the (E) and (Z) isomers of CF₃—CF═CHF; and CO₂.

11. A composition of matter comprising at least one compound of formula (I)
CₓF_{y}—O—CF═CFW (I)
wherein x is 1, 2, 3, 4, 5 or 6 and y=2x+1, and wherein W is F, CF₃, C₂F₅, or C₃F₇ or formula (II)
CₓF_{y}—O—CX═CYZ (II)
wherein x and y have the meaning given above, X stands for H or F, Y stands for H or F and Z stands for H, F, CF₃ or C₂F₅ with the proviso that at least one of X, Y or Z are H and that not more than 2 hydrogen atoms are contained in the compound of general formula (II).
and at least one other organic chemical compound, including azeotropic mixtures and quasi-azeotropic mixtures.

12. The composition of matter according to claim 11, being suitable as a refrigerant or a part of a refrigerant comprising at least one compound of formula (I) or formula (II), wherein the at least one other organic chemical compound is suitable as a component of refrigerants.

13. The composition of matter according to claim 11, consisting of CF₃—O—CF═CF₂ and one or more pentafluoropropenes.

14-18. (canceled)

19. A refrigeration system comprising a refrigerant comprising at least one compound of formula (I)
CₓF_{y}—O—CF═CFW (I)
wherein x is 1, 2, 3, 4, 5 or 6 and y=2x+1, and wherein W is F, CF₃, C₂F₅, or C₃F₇ or formula (II)
CₓF_{y}—O—CX═CYZ (II)
wherein x and y have the meaning given above, X stands for H or F, Y stands for H or F and Z stands for H, F, CF₃ or C₂F₅ with the proviso that at least one of X, Y or Z are H and not more than 2 hydrogen atoms are contained in the compound of general formula (II).

20. The refrigeration system according to claim 19, which is a mobile air conditioning system.

21. The method according to claim 3, wherein the refrigerant comprises at least one non-flammable compound B which eliminates the flammability of the refrigerant.

22. The method according to claim 4, wherein the saturated fluoroether is selected from the group consisting of trifluoromethyl-difluoromethylether (E-125), trifluoromethyl-fluoromethylether (E-134a), and trifluoromethyl-methylether (E-143a).

23. The method according to claim 5, wherein the non-flammable compound B is selected from the group consisting of trifluoromethane, HFC-134, HFC-134a, HFC-125, 1,1,1,3,3-pentafluoropropane (HFC-245fa), 1,1,1,3,3,3-hexafluoropropane (HFC-236fa), 1,1,1,2,3,3,3-heptafluoropropane (HFC-227ea), CF₃—CH═CF₂, (E)-CF₃—CF═CHF, (Z) —CF₃—CF═CHF, and mixtures of the (E) and (Z) isomers of CF₃—CF═CHF.

24. The composition according to claim 11, having a global warming potential (GWP) lower than 150.

25. A composition being suitable as a refrigerant, a heat transformer liquid, a heat pipe liquid, a liquid for an organic Rankine cycle (ORC) process, a solvent blowing agent for foam preparation, an aerosol generating fluid, or a fire extinguishant, said composition comprising a compound of general formula (I) or (II) and optionally comprising a non-flammable liquid or gas,
the general formula (I) being
CₓF_{y}—O—CF═CFW (I)
wherein x is 1, 2, 3, 4, 5 or 6 and y=2x+1, and wherein W is F, CF₃, C₂F₅, or C₃F₇,
or the general formula (II) being
CₓF_{y}—O—CX═CYZ (II)
wherein x and y have the meaning given above, X stands for H or F, Y stands for H or F and Z stands for H, F, CF₃ or C₂F₅ with the proviso that at least one of X, Y or Z are H, and not more than two hydrogen atoms are contained in the compound of general formula (II).

26. The composition according to claim 36, further comprising a non-flammable liquid or gas.
